(19) United States
(12) Patent Application Publication
(10) Pub. No.: US 2010/0131010 A1
(43) Pub. Date: May 27, 2010

(54) EXTRA DISCAL INTERVERTEBRAL STABILIZATION ELEMENT FOR ARTHRODESIS

(76) Inventor: Henry Graf, Lyon (FR)

Correspondence Address: DOWELL & DOWELL P.C., 103 Oronoco St., Suite 220, Alexandria, VA, 22314, US

(21) Appl. No.: 12/452,823

(22) PCT Filed: Jul. 23, 2008
(86) PCT No.: PCT/FR2008/051388

§ 371 Date: Jan. 25, 2010

(30) Foreign Application Priority Data
Jul. 24, 2007 (FR) 0705371
May 6, 2008 (FR) 0802508

(51) Int. Cl. A61B 17/70

(52) U.S. Cl. 606/259; 606/264

(57) ABSTRACT

Extra-discal intervertebral stabilization assembly for arthrodesis, comprising at least two vertebral screws that can engage in two different vertebrae, and a connecting member that is able to connect these two screws, one of each screw or connecting member having a rod (1018), while the other of each screw or connecting member is provided with at least one eyelet (1005) whose walls have a suitable shape, these walls defining an orifice (1004), and the rod or each rod being able to engage in the orifice or each orifice with the possibility of clearance in at least one direction of the plane of this orifice.

The present invention relates to an extra-discal intervertebral stabilization assembly for arthrodesis.

The invention lies in the field of arthrodesis, i.e. bone fusion between at least two adjacent vertebrae. It should be recalled that arthrodesis seeks to allow only micromovements between the vertebrae, and also to damp vibration. Amongst other purposes, such micromovements serve to allow the patient, who can walk again after the operation, to adapt balance as well as possible prior to the bone graft taking.

Typically, and in side view, an extra-discal assembly for arthrodesis of the invention allows movement between two vertebrae of an amplitude that is no greater than about 10% of the natural physiological amplitude. In other words, if the natural maximum amplitude for movement in rotation between two given vertebrae is 10°, then the stabilization assembly in accordance with the invention is suitable for allowing movement of no more than 1° between those two vertebrae.

The stabilization assembly in accordance with the invention is intended to connect together two adjacent vertebrae, while generally being placed on one side only of the vertebral column, i.e. on the right or the left. This stabilization element is implanted in extra-discal manner, i.e. it may be situated at the back or else at the front of the intervertebral space.

The known state of the art relies in particular on plates for interconnecting a plurality of vertebrae, which plates may be fitted to the vertebral bodies using two main placing methods.

Thus, there are screwed plates, such as those designed by Dr. Roy Camille. During surgery, the surgeon puts the plates the vertebrae they are to connect together, and then makes the corresponding securing by inserting a plurality of screws through the plate and the vertebral bodies, in a single step.

As a variant, it is known to begin by fitting pedicular screws in the vertebral bodies of the vertebrae that are to be connected to one another. Once this preliminary step has been performed, a plate is fitted on the free ends of the pedicular screws, and a nut-and-bolt fastening operation is performed. Such a plate of the type that is put into place and then bolted on is described for example in U.S. Pat. No. 4,743,260.

Nevertheless, those various solutions involve certain drawbacks.

It has been found that the screws with which the above-mentioned plates are associated do not possess satisfactory and durable stability relative to the vertebral bodies, because the mechanical stresses induced by the assembly are high. In other words, a certain length of time after implantation, the screws tend to move relative to the vertebral bodies, or even to become separated therefrom.

In addition, those known plates, whether they are of the type that is screwed on or the type that is put into place and then bolted on, do not enable positioning to be achieved that is satisfactory from a physiological point of view. In particular, they give rise to insufficient hollow-back curvature in the patient having the implant.

That said, the invention seeks to remedy those various drawbacks.

To this end, the invention provides an extra-discal intervertebral stabilization assembly for arthrodesis, the assembly comprising:

at least two vertebral screws suitable for penetrating into two different vertebrae; and

a link member suitable for connecting the two screws together;

one of the link member or each screw possessing a rod, while the other of the link member or each screw is provided with at least one eyelet having walls that present a shape that is inherent thereto, said walls defining an orifice, the or each rod being suitable for penetrating in the or each orifice, with freedom to move in at least one direction of the plane of said orifice.

According to other characteristics:

movement is possible in two mutually perpendicular directions in the plane of the orifice, the rod and the walls of the eyelet forming a hinge only when that one of the rod and the eyelet that is carried by the link member puts under tension that one of the eyelet or the rod that is carried by the screw;

the hinge acts via a single point of contact between the rod and the walls of the eyelet.

the hinge acts via a contact of the flat-on-flat type, so as to make sub-luxation possible;

the rod is free to move relative to the walls of the eyelet in a single direction in the plane of the eyelet, so as to form a slideway connection between the rod and the eyelet;

at least one eyelet presents rigid walls;

at least one eyelet presents a wall that is deformable, at least in places, under the effect of a stress that is considerably greater than that due to gravity;

at least one eyelet is provided with means for resisting movement of the rod;

the means for resisting movement comprise a deformable narrowed wall of the eyelet;

the means for resisting movement comprise a partial filling of the orifice defined by the eyelet by means of an elastomer material;

the link member comprises an elongate body and two sleeves suitable for being fitted on said body, each sleeve being provided with a corresponding eyelet;

a first sleeve is stationary relative to the body, while a second sleeve is movable relative to the body;

at least one spring is provided interposed between the movable sleeve and the stationary sleeve, and/or between the movable sleeve and an end abutment of the elongate body;

the link member is formed by a single link element presenting two eyelets, each eyelet being suitable for receiving a rod carried by a vertebral screw;

the link member is formed by two distinct link elements, each link element being provided with two eyelets, each rod of a vertebral screw being suitable for penetrating in two successive eyelets, carried respectively by the two distinct link elements;

a first spring is interposed between the movable sleeve and the stationary sleeve of a first link element, while a second spring is interposed between the movable sleeve and the end abutment of the second link element;

when the rod bears against an axial end of the eyelet, the main axis of the rod being perpendicular to the plane of the eyelet, the screw and the eyelet define a free zone that is not occupied by the rod, said free zone presenting a dimension along the main axis of the eyelet that is greater than or equal to 50%, in particular greater than or equal to 100%, of the dimension of the rod measured along the same main axis;

the body of the link member possesses shape that is inherent thereto;

the body is made as a single piece;

the body is made of two segments, each provided with a corresponding orifice, and an intermediate element, in particular of the damper type, is interposed between the two segments;

at least one orifice lies adjacent to an end rim;

at least one orifice lies adjacent to an abutment wall forming part of a central block;

the body possesses length lying in the range 15 millimeters (mm) to 45 mm, and width lying in the range 5 mm to 10 mm;

each pedicular screw possesses a rod of cross-section that is smaller than the cross-section of the orifice through which it extends, so as to form clearance in two directions in the plane of the body of the stabilizer element;

the rod is provided with at least one head of elongate shape, presenting a length that is shorter than the length of the oblong orifice, while being longer than the width of said oblong orifice, and the width of said head is less than the width of the orifice;

the head is formed by a portion of a sphere that is truncated by two flats;

the rod is provided with a through opening suitable for receiving a cotter pin that is to come into abutment against the facing walls of the oblong orifice;

the rod is threaded and co-operates with a nut against which the walls of the orifice are suitable for bearing;

the rod of the screw is terminated by a spherical head, the screw being suitable for extending through an orifice made up of a circular main portion that is extended by two notches; and

the nut comprises a head made of a damper material suitable for coming into abutment against the rim and/or the abutment wall of the extra-discal element.

The invention also provides a method of fitting the above assembly, the method comprising the following steps:

implanting the at least two vertebral screws in respective vertebrae;

inserting the free ends of the screws through orifices in the or each link member; and

putting the or each link member under tension so as to exert a corresponding tension force on the vertebral screws.

According to other characteristics:

the at least two screws are implanted in respective vertebrae in such a manner that the distance between the free ends of the screws is different from the distance between the centers of the orifices in the link member; the distance between said free ends is then modified, by applying external action, in particular with the help of a tool, so that said distance becomes close to the distance between the centers of the orifices; and finally the free ends are inserted through the orifices, after which the external action is released so as to put the or each link member under tension:

an external action tending to move the screws towards each other is applied, and then when said action is released, the screws come into abutment against the further-apart walls of the orifices so as to place the link member under tension so that it acts as a guy;

an external action is applied that tends to move the screws apart from each other and then, when said action is released, the screws come into abutment against the closer-together walls of the orifices, so as to place the link member under tension, acting as a stay;

the same screws are connected together by means of two different link members, a first link member being put into place adjacent to the vertebral bodies under tension to act as a stay, and a second link member is put into place remote from the vertebral bodies under tension to act as a guy; and

a transverse link member is used to connect a pedicular screw implanted on a first, right or left, side of a first vertebral body with another pedicular screw implanted in the opposite, left or right, side respectively of a vertebral body that is immediately adjacent to said first vertebral body.

The invention is described below with reference to the accompanying drawings, given purely by way of non-limiting example, and in which:

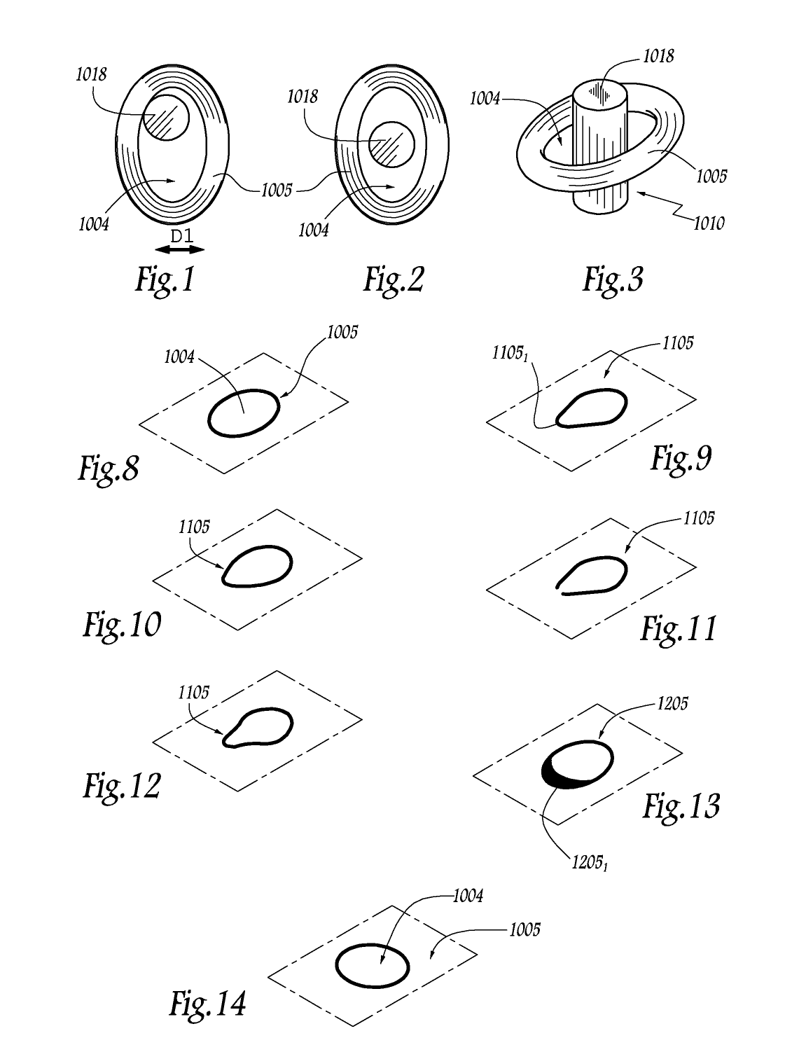

FIGS. 1 and 2 are plan views showing an eyelet and a rod forming part of an extra-discal assembly in accordance with the invention;

FIG. 3 is a perspective view showing the eyelet and the rod;

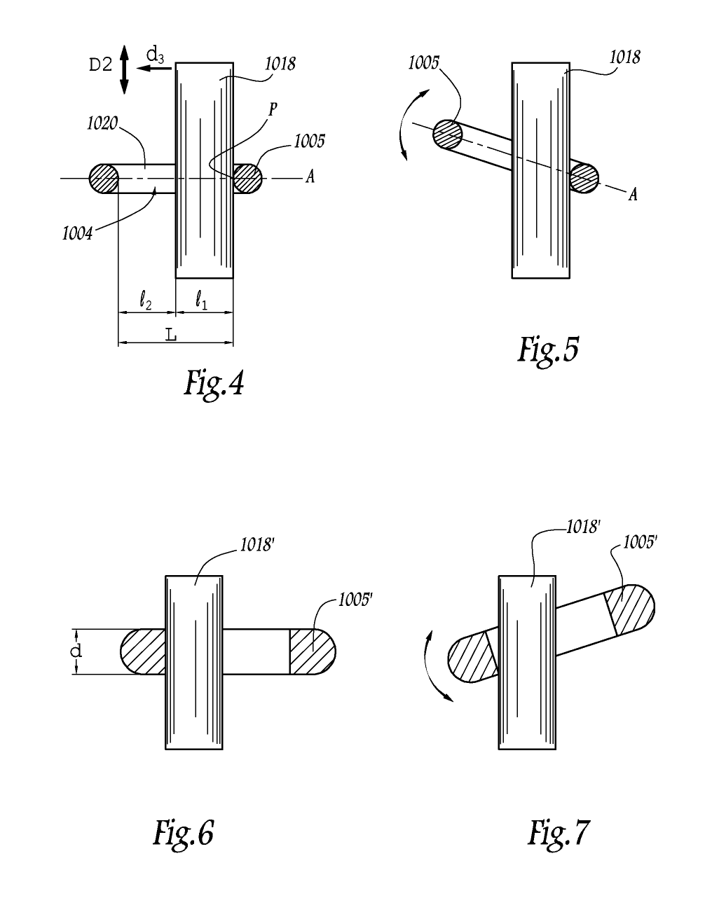

FIGS. 4 and 5 are side views showing the relative freedom of movement between the eyelet and the rod;

FIGS. 6 and 7 are side views, analogous to FIGS. 4 and 5, showing a variant embodiment of the eyelet;

FIGS. 8 to 14 are views diagrammatically showing respective other variant embodiments of the eyelet;

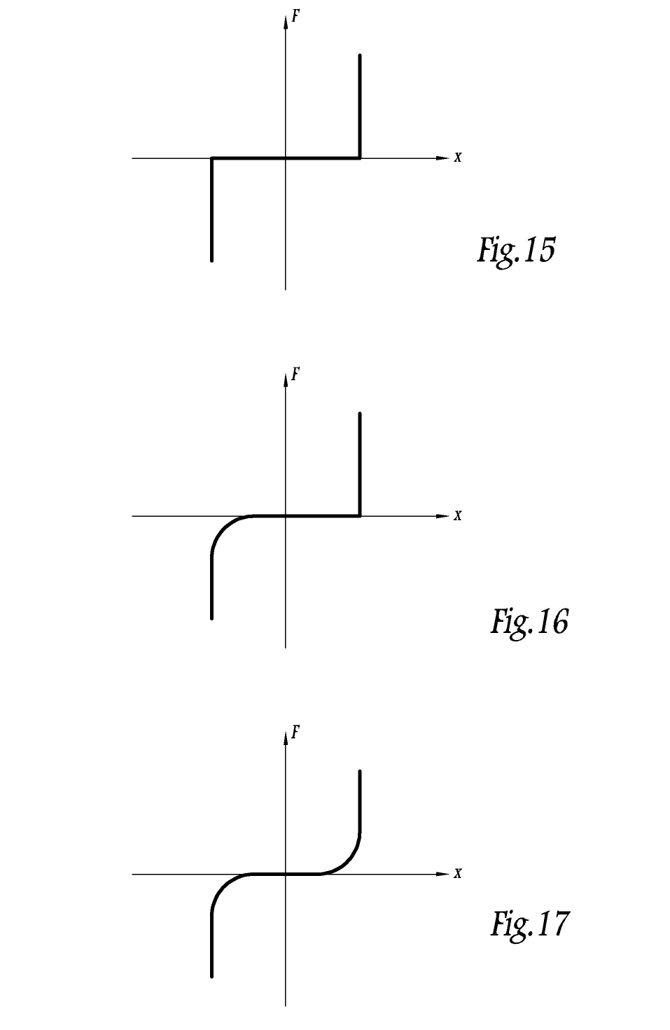

FIGS. 15 to 17 are graphs showing force as a function of the movement of the rod along the eyelet, for some of the above embodiments;

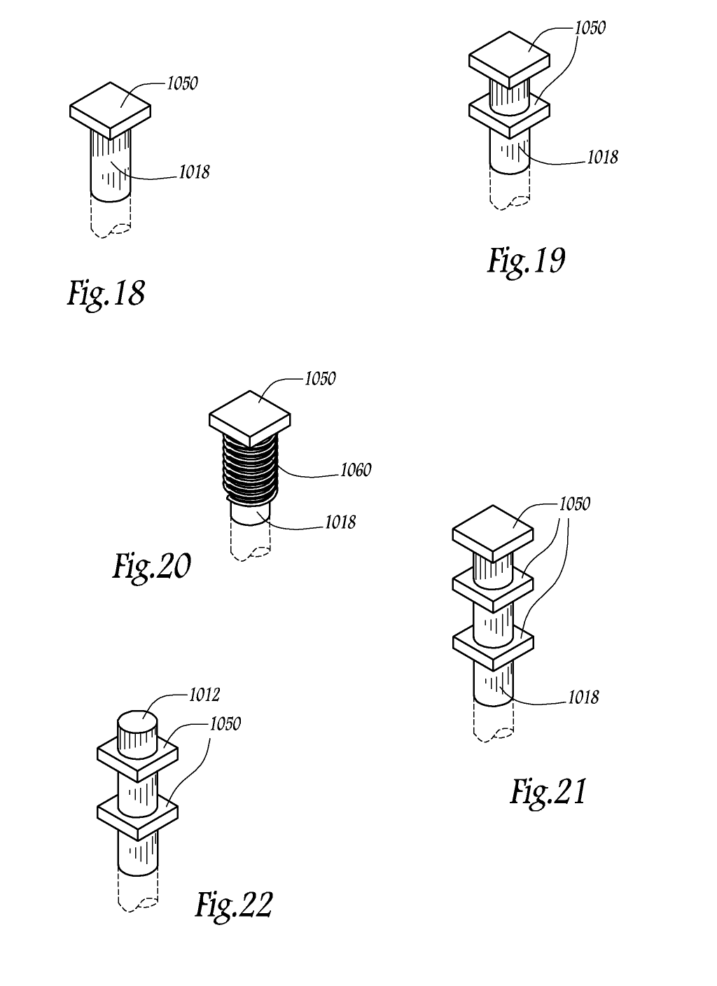

FIGS. 18 to 22 are perspective views showing different variant embodiments of the rod;

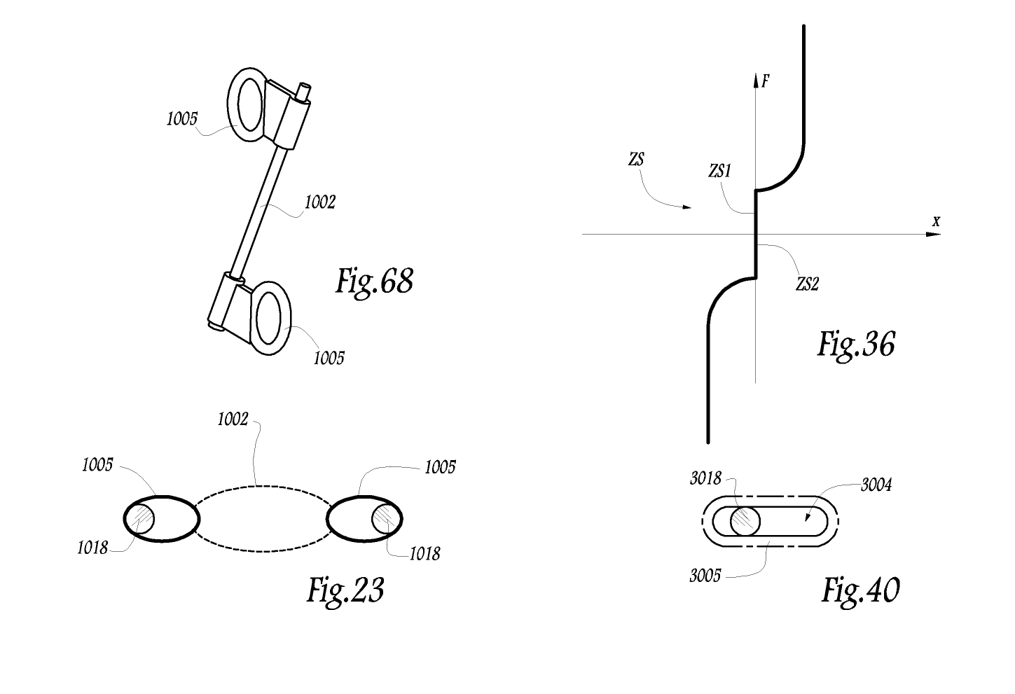

FIG. 23 is a general diagrammatic view showing the extra-discal assembly in accordance with the invention;

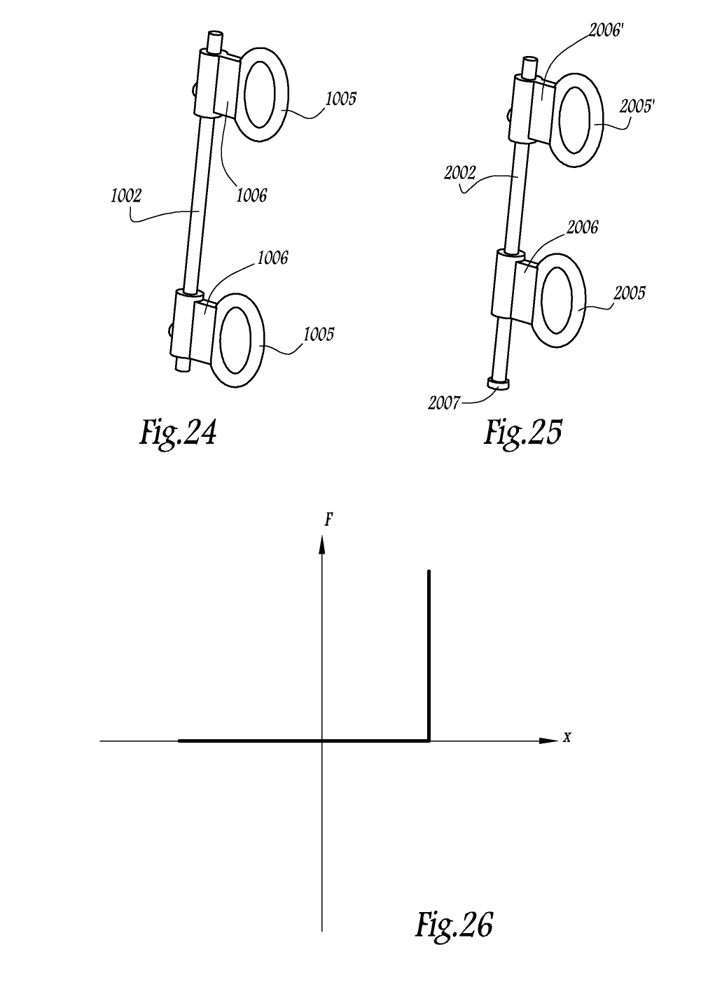

FIG. 24 is a perspective view showing a variant embodiment of the invention;

FIG. 25 is a perspective view showing an arrangement that does not form part of the invention;

FIG. 26 is a graph in which the curve shows force as a function of movement, for the arrangement of FIG. 25;

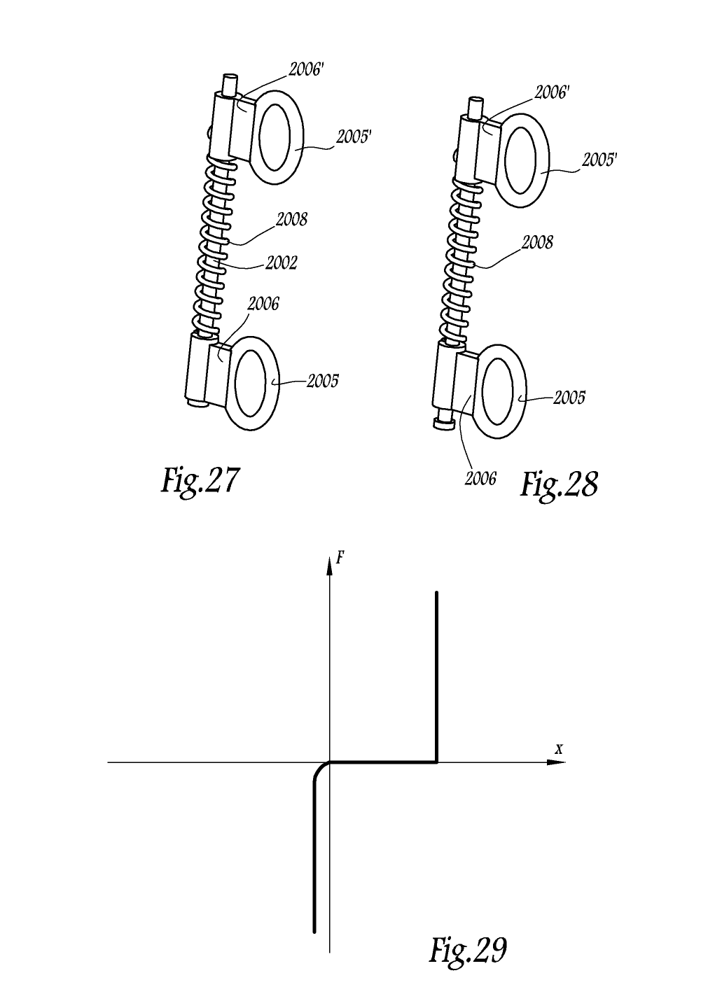

FIGS. 27 and 28 are perspective views showing an additional arrangement that does not form part of the invention, in two positions;

FIG. 29 is a graph analogous to FIG. 26 relating to the arrangement of FIGS. 27 and 28;

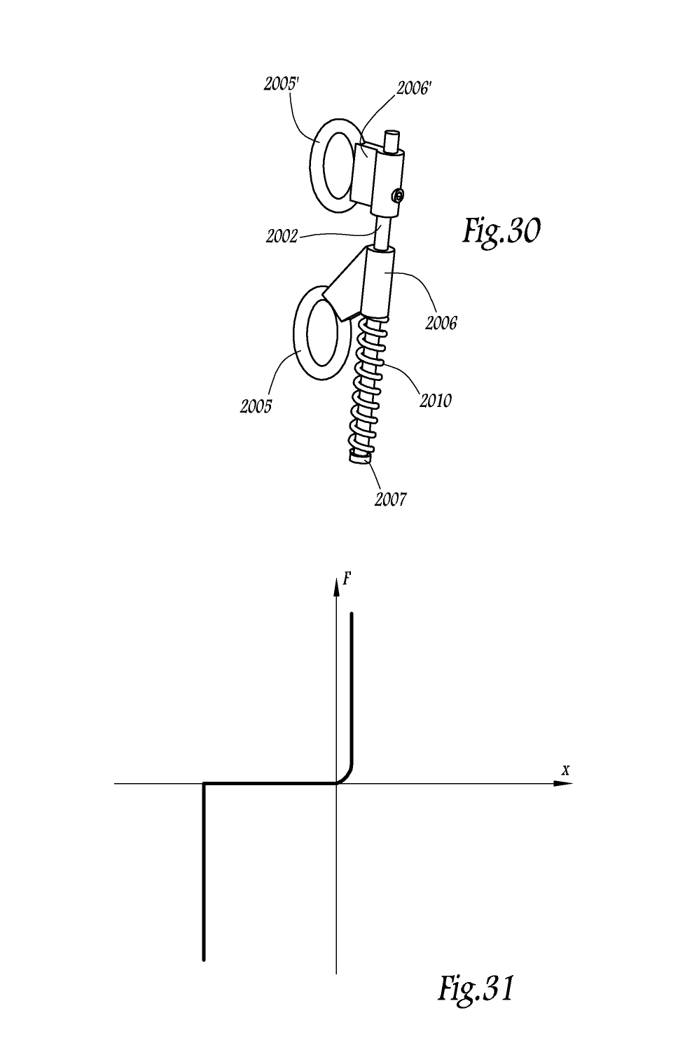

FIG. 30 is a perspective view showing an additional variant that does not form part of the invention;

FIG. 31 is a graph analogous to FIG. 26 relating to the FIG. 30 arrangement;

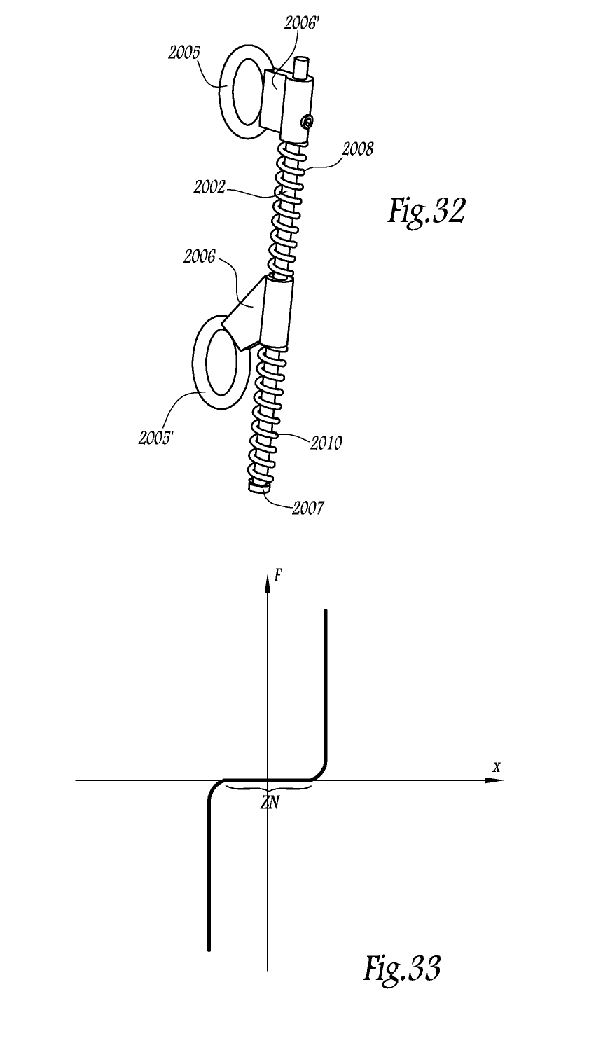

FIG. 32 is a perspective view showing an additional arrangement that does not form part of the invention;

FIG. 33 is a graph analogous to FIG. 26 relating to the FIG. 32 arrangement;

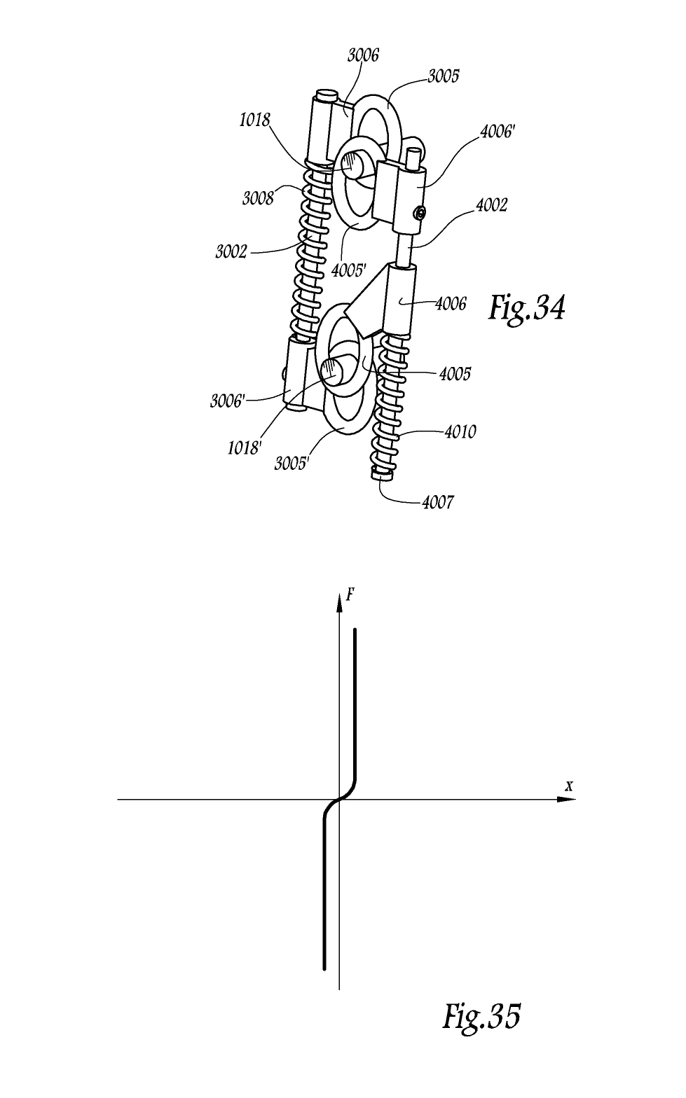

FIG. 34 is a perspective view showing an additional variant embodiment of the invention;

FIG. 35 is a graph analogous to FIG. 26 relating to the embodiment of FIG. 34.

FIG. 36 is a graph analogous to FIG. 26 relating to another embodiment of the invention (not shown);

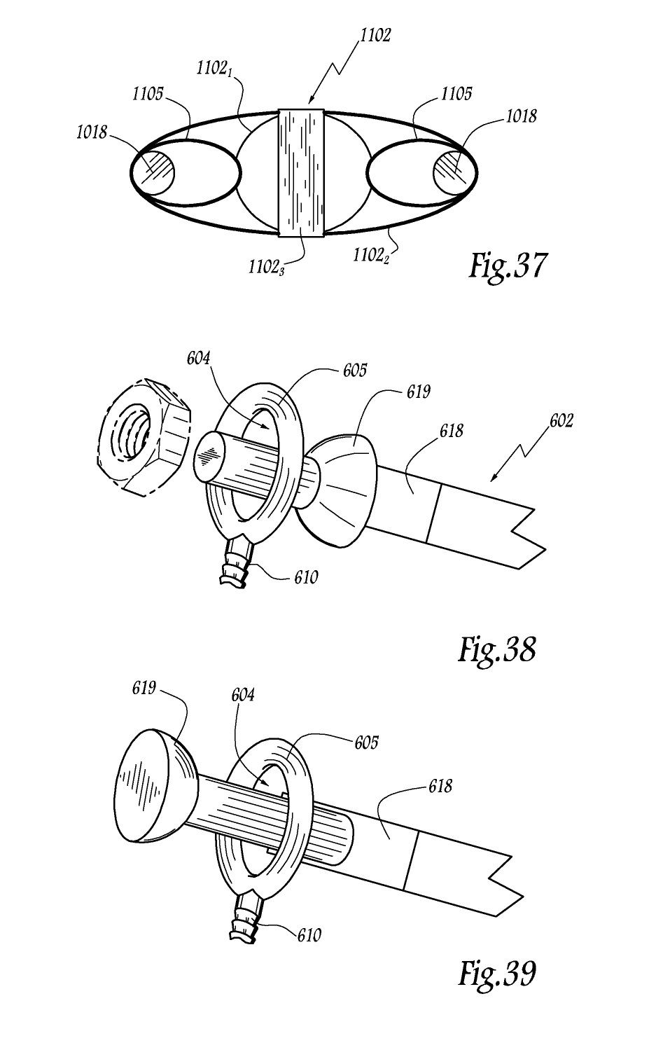

FIG. 37 is a side view showing an additional variant of the invention;

FIGS. 38 and 39 are perspective views showing two additional variants of the invention;

FIG. 40 is a diagrammatic view showing an additional variant embodiment of a rod and an eyelet in accordance with the invention;

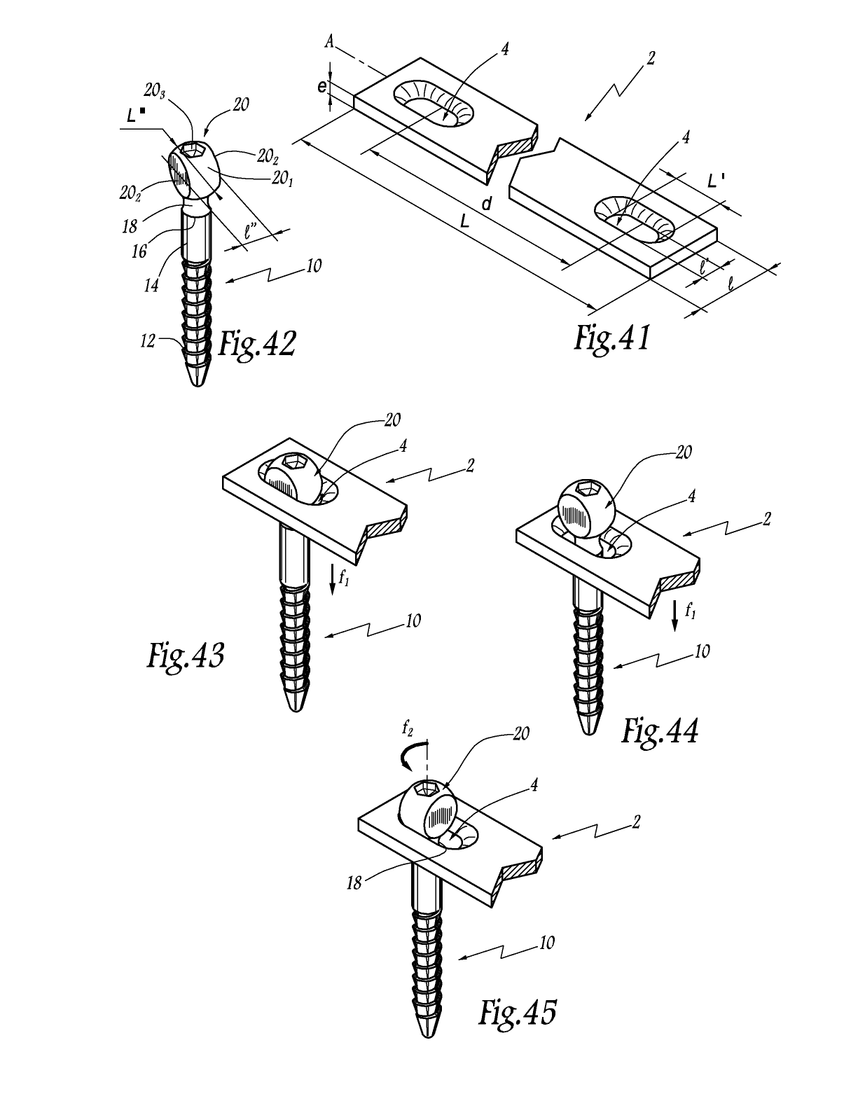

FIG. 41 is a perspective view showing a link member forming part of the extra-discal assembly in accordance with the invention;

FIG. 42 is a perspective view showing a pedicular screw suitable for being associated with the member of FIG. 41;

FIGS. 43 to 45 are perspective views showing said screw secured with said link member;

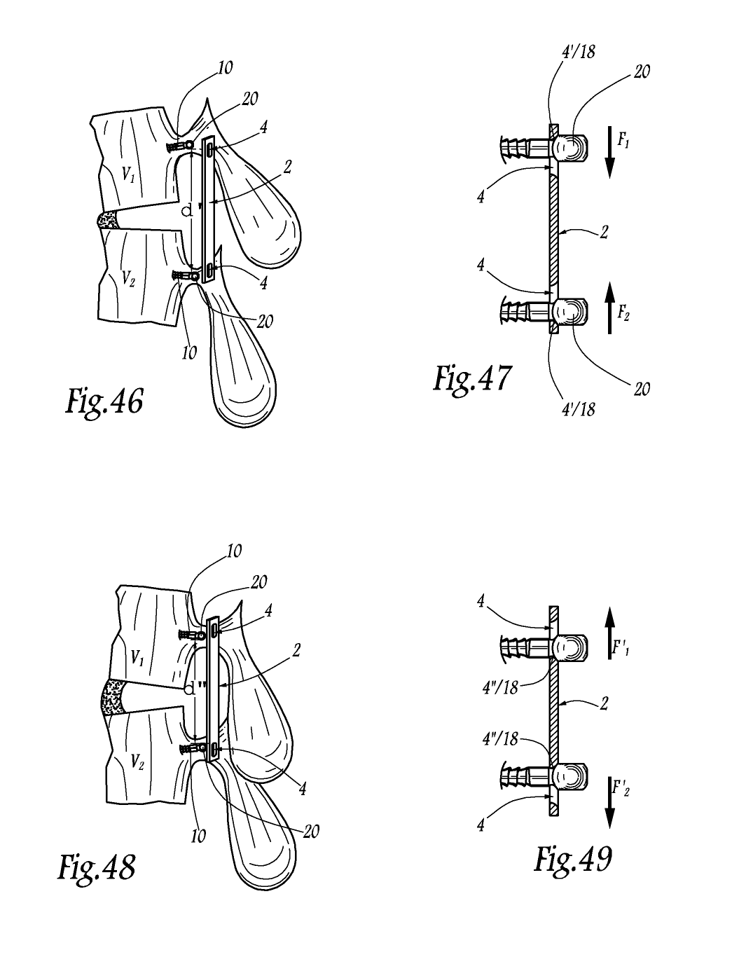

FIGS. 46 and 47 are side views showing a first type of implantation for the link member in accordance with the invention;

FIGS. 48 and 49 are side views showing another type of implantation for the link member in accordance with the invention;

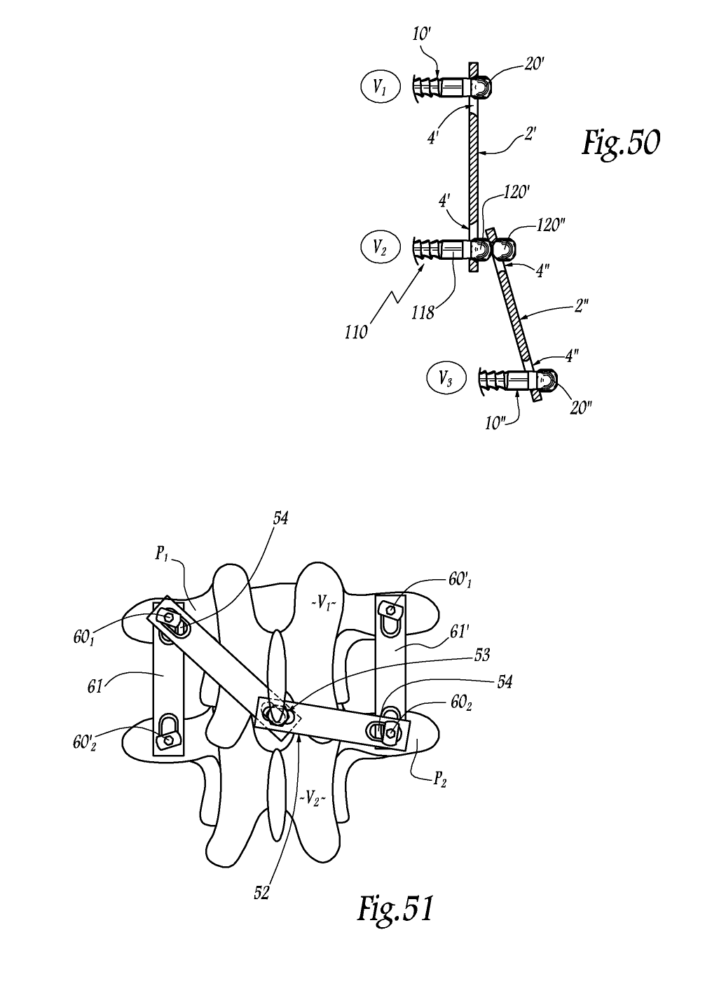

FIGS. 50 and 57 are longitudinal section views showing two variant embodiments of the invention;

FIG. 51 is a rear view showing an additional variant embodiment of the invention;

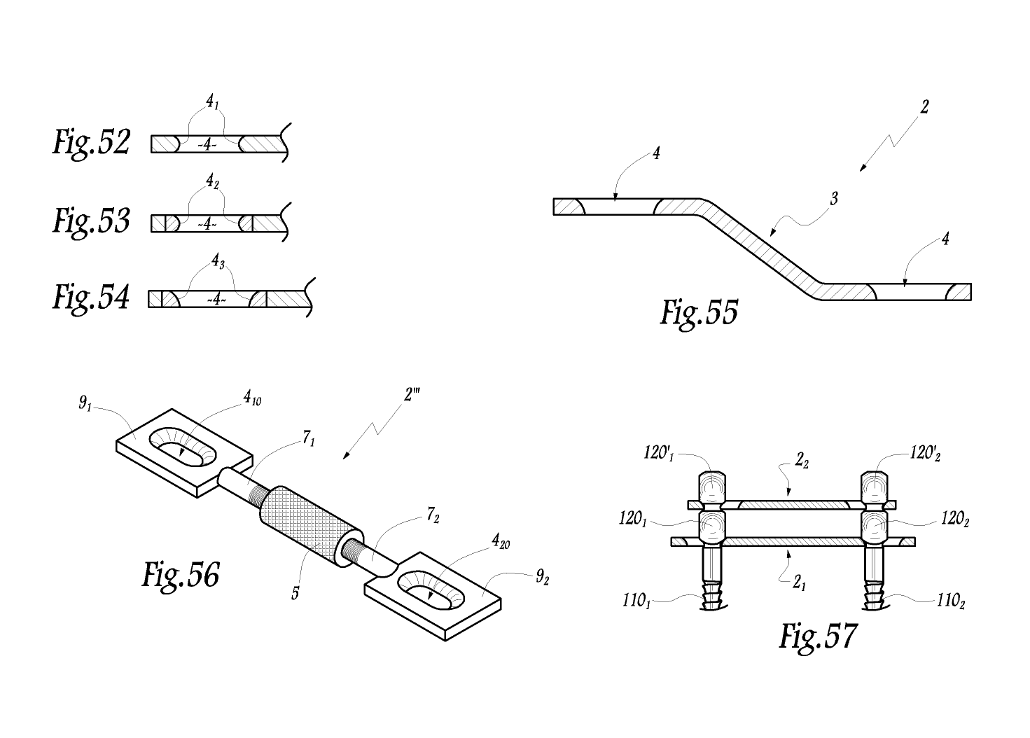

FIGS. 52 to 54 are longitudinal section views showing various different profiles for the link member in accordance with the invention;

FIG. 55 is a side view showing another variant embodiment of the intervertebral link member;

FIG. 56 is a perspective view showing another variant embodiment of a link member in accordance with the invention;

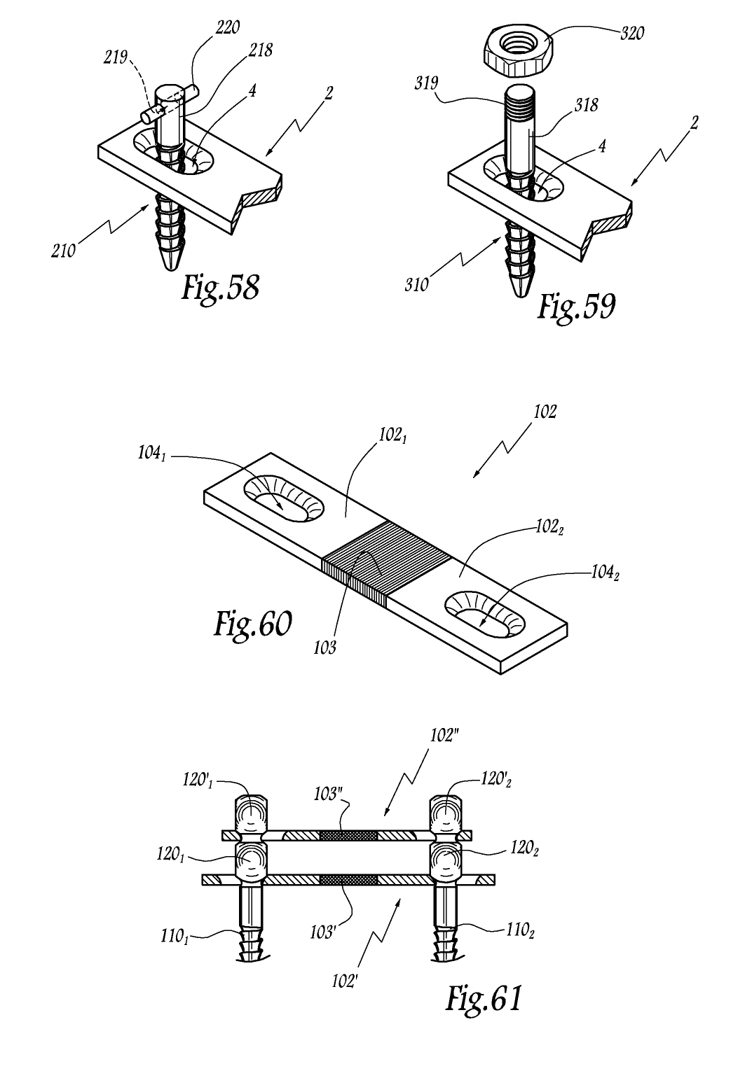

FIGS. 58 and 59 are perspective views showing two different embodiments of a pedicular screw for associating with a link member in accordance with the invention;

FIG. 60 is a perspective view showing an additional variant embodiment of the link member in accordance with the invention;

FIG. 61 is a longitudinal section view analogous to FIG. 57, showing an additional variant embodiment of the invention;

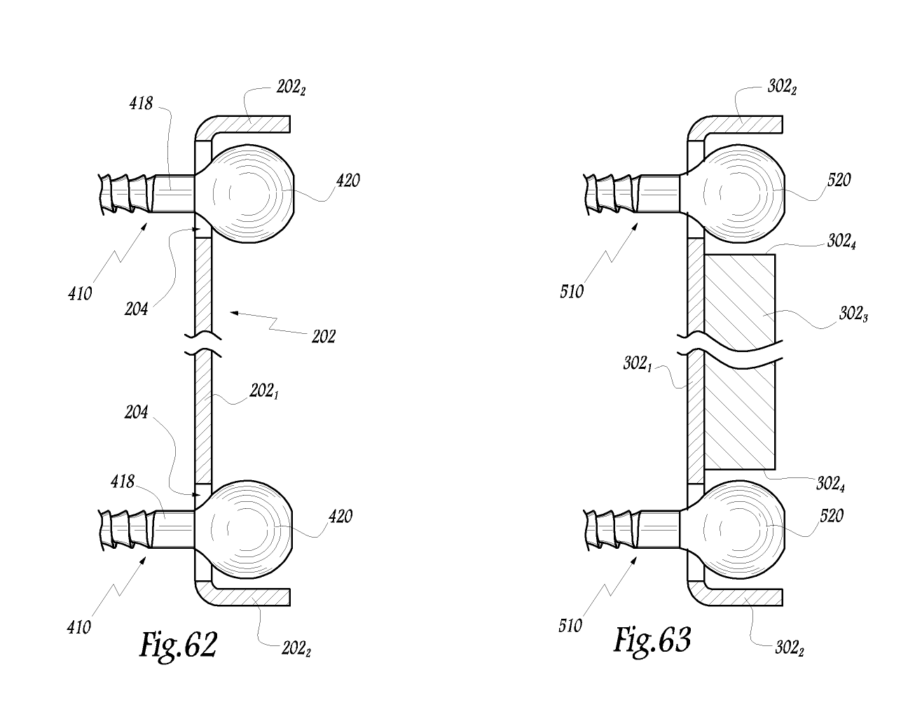

FIGS. 62 and 63 are longitudinal section views showing two other variant embodiments of the link member in accordance with the invention;

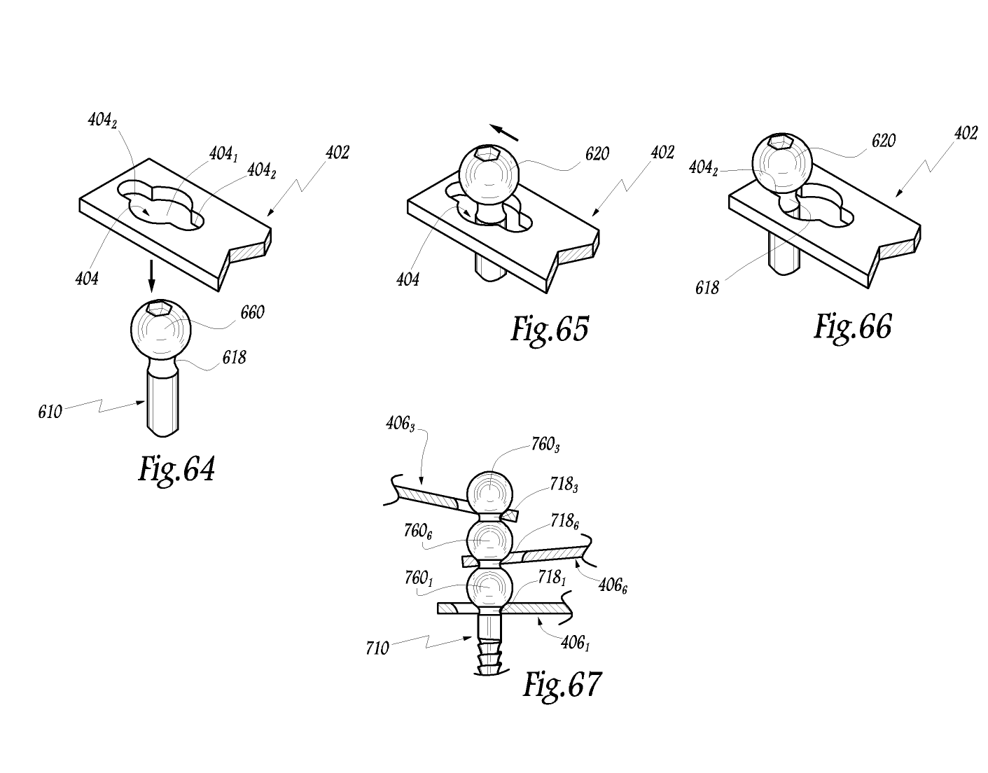

FIGS. 64 to 66 are perspective views, analogous to FIGS. 43 to 45 showing the mounting of a pedicular screw and a stabilizer element, both in accordance with an additional variant of the invention;

FIG. 67 is a fragmentary section view showing an alternative embodiment of FIGS. 64 to 66; and

FIG. 68 shows a variant of the embodiment of FIG. 24.

FIGS. 1 to 3 illustrate the subject matter of the invention in all its generality.

The invention may be generalized to using two eyelets, one of which, referenced 1005, is shown in these figures, the eyelet defining two oblong orifices, one of which 1004 is shown. Each eyelet presents a shape that is inherent thereto, i.e. its shape does not vary under the effect of gravity or under the effect of other stresses of analogous magnitude. In contrast, each eyelet is capable of deforming, at least in certain places, under the effect of stresses of magnitude that is significantly greater than gravity, as described in more detail below.

FIGS. 1 to 3 show more particularly the co-operation between the eyelet 1005 and a rod 1018 forming part of a screw 1010 and that penetrates in the orifice 1004 as defined by the eyelet 1005. By way of example, the screw 1010 is a pedicular screw that includes, in conventional manner, a threaded zone for penetrating into a vertebral body. However, provision may be made to use not a screw of pedicular type, but some other type of vertebral screw.

Thus, the screw is suitable for being implanted in a vertebral body, either laterally or anteriorly, or in a vertebral body through the pedicle. In general, any insertion may be used that ensures the screw is secured stably to the vertebra. It is then implanted in the vertebra by a screw thread and outside the vertebra it presents a projecting stud that co-operates with a link element, as described below. The stud may also be supported by a mechanical member other than a screw thread, such as for example a staple or hooks that are placed on the vertebral body and/or the intervertebral bone plates.

In FIGS. 2 and 3, which correspond to there being no tension applied by the link element to the screw, the rod penetrates in the orifice in loose or floating manner. In other words, there exist three degrees of freedom in rotation between the screw and the eyelet, and also two degrees of freedom in translation, in two mutually perpendicular directions in the plane of the eyelet. In addition, in the absence of blocking means, there is a third degree of freedom in translation for the rod relative to the eyelet, in a direction that is perpendicular to the plane of the eyelet.

It is now assumed that an external action is exerted on the screw, in particular under the effect of certain movements made by the patient. This action causes relative movement between the eyelet and the rod, which in turn causes the rod to come into abutment against the walls of the eyelet.

This puts the eyelet into tension against the rod, which tension presents a component acting in the plane of the eyelet, i.e. the plane of the sheet of FIG. 1. This coming into abutment, accompanied by the application of a certain amount of tension, creates a hinge between the rod and the walls of the eyelet.

As shown in FIG. 4, which roughly corresponds to FIG. 1, the walls of the eyelet advantageously present a profile that is curved when seen in cross-section, such that the hinge formed in this way is a point hinge. The corresponding point of contact is referenced P. When tension is applied in this way, the rod and the walls of the eyelet continue to present three degrees of freedom in rotation relative to each other, as shown in particular in FIG. 5. In addition, there is still one degree of freedom in translation along the angle of inclination arrow D1 in FIG. 1, and one degree of freedom in translation along the vertical arrow D2 in FIG. 4.

In contrast, and with reference to FIG. 4, the freedom of the rod to move to the left is limited by the facing walls of the orifice. Under such conditions, there exists only “half” a degree of freedom in translation along angle of inclination arrow d3 in FIG. 4. In order to represent this half-degree of freedom in translation, the arrow is single-headed, unlike the other two “full” degrees of freedom in translation which are represented by double-headed arrows D1 and D2.

FIGS. 4 and 5 also show the relative dimensions of the rod 1018 and the orifice 1004. The size of the orifice measured along the axis A connecting the eyelet 1005 with the other eyelet (not shown) is written L. In other words, the axis A corresponds to the main axis of the link member, interconnecting the two screws 1010.

The size of the screw, likewise along the axis A, is written [symbol] . In the position of FIG. 4, when the rod bears against a first end of the orifice, it defines a free zone 1020 of the orifice 1004, which zone corresponds to the region that is not occupied by the rod. The size of this free zone along the axis A is written [symbol] , it being understood that L= [symbol] ₁+ [symbol] ₂.

Advantageously, when the rod 1018 extends perpendicularly to the plane of the eyelet, as shown in FIG. 4, [symbol] ₂ is greater than or equal to [symbol] ₁/2, and preferably [symbol] ₂ is greater than or equal to [symbol] ₁. This makes it possible firstly to enable the rod to move along the eyelet during mutual rotation between the rod and the eyelet, as shown in FIG. 5. In this respect, it should be observed that in accordance with the invention, this possibility for movement in rotation between the rod and the eyelet is always allowed.

This dimension for the free zone 1020 also makes it possible for the rod to move towards the left, as would happen under the effect of the patient performing a movement of large amplitude. When the link member acts as a guy, such a large-amplitude movement corresponds to hyperextension, whereas when the link member acts as a stay, the movement corresponds to hyperflexion.

It should be observed that in prior art link members, in particular plates, there necessarily exists a certain amount of assembly clearance for the screw in the plate. Nevertheless, such clearance cannot be considered as acting like the free zone explained with reference to FIGS. 4 and 5, insofar as it does not present a dimension of sufficient size to enable the above-mentioned functions to be performed.

It should be observed that the rod and the eyelet may present shapes that differ from those shown in the above figures. Thus, the rod may present a section that is not circular, e.g. that is square, rectangular, or some other shape. Furthermore, the walls of the eyelet may define any suitable shape, for example a circle, an oval, a lozenge, or indeed a more complex shape.

As an additional variant, shown in FIGS. 6 and 7, the hinge between the rod 1018’ and the walls of the eyelet 1005′ may be achieved via contact that is not of the point type but rather of the flat-on-flat type. These two flats define a contact zone that is relatively small, represented by the distance d, thus making it possible for sub-luxation of the rod relative to the walls of the orifice (arrow F). In other words, there is a hinge that can be considered as acting like the hinge of FIGS. 4 and 5.

FIGS. 8 to 14 show various possibilities for the shape of the walls of the orifice. In these figures, the body of the eyelet is represented in highly diagrammatic manner, by chain-dotted lines.

In FIGS. 8 and 14, the orifice is of oval shape, while extending in one or the other of the main directions of the plane of the eyelet. In other words, compared with FIG. 8, the walls of the orifice in FIG. 14 are turned through one-fourth of a turn.

FIG. 9 shows an additional variant of the invention in which at least one eyelet is not rigid, but presents a shape that is inherent thereto, as defined above. More precisely, the eyelet 1105 presents an end zone 1105₁ that is narrowed.

Thus, the rod is capable of moving initially in a zone of the eyelet that presents transverse dimensions that are greater than those of the rod, such that the movement takes place without effort. Then, in the vicinity of the narrowed zone, the rod can still move, but only because of the deformable nature of the eyelet. Such movement therefore takes place against mechanical resistance, and it is possible to modulate the magnitude thereof. This is advantageous since it provides means for unilateral damping, provided solely by means of the eyelet in association with its composition and/or its geometry.

FIGS. 10 and 12 show a variant of the FIG. 9 embodiment. In these two additional figures the end zone of the eyelet is narrowed to a greater or lesser extent.

FIG. 11 shows an additional variant of the invention, similar to that of the preceding figure, in which the eyelet does not define a closed loop. Thus, it presents a shape that is generally oval, together with a break zone of small dimensions, so as to define two facing free ends.

As in the preceding embodiment, in the absence of any stress, these two free ends define a narrowed zone, of transverse dimension that is smaller than that of the rod. As a result, when the rod moves, it can splay these two ends apart against a given resistance, until it comes into abutment against the free ends. Naturally, these ends are designed so as to ensure that the rod cannot escape from the inside volume of the eyelet.

In an additional embodiment (not shown), the eyelet may be provided with a spring blade suitable for pivoting about a hinge that is generally perpendicular to the main plane of the eyelet. Under such conditions, movement of the rod takes place against a predefined resistance provided by the spring blade. Thereafter, when the rod returns to its initial high position, the spring blade also returns to its initial position.

FIG. 13 shows an additional variant embodiment of the invention in which the end of the eyelet 1205 is provided with a filling material of elastomer nature, such as rubber 1205₁. In this way, when the rod moves downwards in this figure, this movement takes place against the resistance imparted by the elastomer material.

As can be seen from the above, the embodiments of FIGS. 9 to 13 involve a certain amount of deformation of the walls of the orifice. Nevertheless, given that the present invention is situated in the context of arthrodesis, this elasticity is limited, so as to allow only micromovements between the vertebrae, and also so as to damp vibration.

FIG. 15 is a graph showing force F as a function of movement referenced x. In other words, if it is assumed that the eyelet is stationary, then the curve represents the force required to move the rod along the eyelet.

In FIG. 15, the curve relates to the embodiment of FIGS. 1 to 3 in which the inside volume of the eyelet is empty and the eyelet presents rigid walls. In other words, this curve is made up of three segments, namely firstly a horizontal middle segment corresponding to the rod moving between the two facing walls of the eyelet, with this taking place without resistance, i.e. requiring zero force. The middle segment lies between two end segments that are vertical. In other words, when the rod reaches one or the other of the walls of the eyelet, it can no longer move regardless of the value of the applied force.

FIG. 16 shows the same curve, but for an eyelet having one end that is rigid and the other end that offers resistance using one or other of the configurations described by way of example in the preceding figures (narrowed wall, hinge, or elastomer material).

Once more there is a horizontal middle zone, that is shorter than the middle zone in the preceding figure, and that corresponds to the rod moving between the rigid wall and the means that provide resistance. Beside the rigid wall (to the right), the horizontal segment is terminated by a vertical segment as in the preceding example. In contrast, at the opposite side (to the left), the horizontal segment is extended by a segment presenting a more or less exponential shape, that is nevertheless associated with an asymptote, corresponding to the movement limit for a theoretically infinite force.

FIG. 17 shows a curve relating to an eyelet having both ends associated with means that enable a resistance to be exerted against movement of the rod, as in the right-hand portion of the preceding graph. The associated curve thus presents a horizontal segment that is shorter than that of the preceding figures, corresponding to the rod moving between the two resisting means on either side of the eyelet. This horizontal segment, referred to as a “neutral zone”, is extended to right and to left by two segments that are analogous to that of the preceding figure, each being associated with an asymptote.

FIGS. 18 to 22 show various possible embodiments of the invention. In these figures, there can be seen the rod 1018, together with various abutments 1050 that enable the movement of each eyelet along the rod to be limited. It should be observed that these figures do not show the eyelet.

In FIG. 18, there is provided a single end abutment 1050. In addition, in FIG. 20, the single end abutment co-operates with a spring 1060 that is interposed between said abutment and the facing walls of the eyelet (not shown). The end abutment may be stationary, or it may be mounted to slide on the rod.

In FIGS. 19 and 22, there is provided an end abutment together with an intermediate abutment, thereby enabling two eyelets to be engaged on the same rod. Once more, the abutment may be stationary, or it may be slidably mounted on the rod.

Finally, FIG. 21 provides an end abutment together with two intermediate abutments, thus enabling three eyelets to be engaged on the same rod. As before, each abutment may be stationary or may be slidably mounted on the rod.

In these FIGS. 18 to 22, each abutment is made in the form of a plate. Nevertheless, provision could be made to confer other shapes to one or more of these abutments, for example they could be round, oval, or of some other shape.

The link member forming part of the stabilizer assembly of the invention comprises, in very general terms, firstly two eyelets 1005 as described above, and secondly a middle zone 1002 extending between the two eyelets, as shown diagrammatically in FIG. 23. This middle zone may be rigid or substantially rigid, or indeed it may present a shape that is inherent thereto, as defined above. It is also possible to provide for it to be a flexible or resilient zone. It is also possible to use a middle zone that has various combinations of these characteristics, i.e. rigid, of inherent shape, flexible, and resilient.

FIG. 24 shows an advantageous possibility in which an elongate link body 1002 is provided that is implemented by way of example in the form of a rigid tube. Each eyelet 1005 is associated with a link sleeve 1006 suitable for sliding about the link tube. Each eyelet may thus be secured by any appropriate means relative to the tube 1002, with it being possible to adjust its position along the main direction of the body 1002. In other words, the distance between the two eyelets may be adjusted very accurately in situ, in particular by the surgeon while performing the operation.

FIGS. 25 to 33 show arrangements that do not form part of the invention. Nevertheless, it is informative to describe these “intermediate” type arrangements in order to better understand the embodiment of FIG. 34, which is indeed in accordance with the invention.

FIG. 25 shows a first intermediate arrangement that differs from the embodiment of FIG. 24 in that one of the sleeves, the sleeve 2006, is mounted to slide freely relative to the link tube 2002 until it reaches a terminal abutment 2007 thereof, whereas the other sleeve 2006′ is stationary. The curve plotting force as a function of movement (FIG. 26) begins with a vertical segment corresponding to the sliding sleeve coming into abutment against the end of the link tube. Thereafter there is a horizontal segment that corresponds initially to the rod moving without resistance between the walls of the eyelet, followed by the eyelet moving as a result of the rod moving, the eyelet likewise moving freely along the link tube.

With reference to FIGS. 27 and 28, it is now assumed that a spring 2008 is interposed between the two sleeves, respectively the stationary sleeve 2006′ and the sliding sleeve 2006. The corresponding curve (FIG. 29) then has a vertical segment as in the preceding figure, followed by a horizontal neutral zone corresponding to the rod moving freely along the sliding eyelet. If the movement of the rod is continued towards the stationary eyelet, then the movement takes place against the spring. Under such conditions, the horizontal neutral zone is extended by a segment of exponential appearance, associated with an asymptote, of the same type as in the figures showing an eyelet that presents resistance means (narrowed walls, elastomer, . . . ).

In FIG. 30, a spring 2010 is interposed between the facing walls of the abutment and the sliding sleeve. The corresponding curve (FIG. 31) has a horizontal neutral zone corresponding to the top sleeve sliding freely along the tube between the stationary sleeve and the spring, and also to the rod moving freely in the free eyelet. Thereafter, there is a segment of exponential appearance associated with an asymptote, corresponding to the free sleeve moving against the spring.

In FIG. 32, two springs are provided, one of the springs 2010 being interposed between the abutment 2007 and the free sleeve 2006, and the other spring 2008 being interposed between the free sleeve and the stationary sleeve 2006′. The corresponding curve (FIG. 33) then comprises a horizontal neutral zone ZN corresponding to the rod moving freely between the two ends of the moving eyelet. Thereafter, there are two segments of exponential appearance, each associated with an asymptote, corresponding to the moving sleeve moving against a respective one of the two springs.

FIG. 34 shows an embodiment that is in accordance with the invention. With reference to this figure, it is assumed that the link member is no longer formed by a single link member, as in the various above embodiments, but rather by two link elements 3002 and 4002. In other words, these two link elements define four eyelets 3005, 3005′, 4005, and 4005′, with each rod 1018 and 1018′ penetrating in succession through two of the eyelets.

These link elements can be dimensioned so that each rod comes into abutment firstly against the first side of the walls of the eyelet of the first element, and secondly against the opposite side of the walls of the eyelet of the second element. Under such conditions, if there is no possibility for the rod to move, and if it is assumed that the walls of the eyelet are rigid and that the eyelets are stationary relative to the elongate link body, then there is no possibility of moving the rod. In other words, the associated curve (not shown) would then consist in a single vertical segment coinciding with the ordinate axis. This embodiment (not shown) presents certain advantages insofar as it provides hyperstability to the link between the two vertebrae it connects together.

The embodiment of FIG. 34 provides a possibility for relative movement between the rods. For this purpose, each link element possesses a stationary sleeve and a sliding sleeve, as in the embodiment of FIG. 25.

In addition, as in the embodiment of FIGS. 27 and 28, the first link element is associated with a spring 3008 interposed between the two sleeves 3006 and 3006′. In addition, as in FIG. 30, the second link element is associated with a spring 4010 interposed between an end abutment and the sliding sleeve 4006.

In the equilibrium position of FIG. 34, it can be seen that the rods come into abutment against the adjacent walls of the two eyelets 3005 and 3005′. In contrast, these rods come into abutment against the opposite walls of the other two eyelets 4005 and 4005′.

Starting from FIG. 34, if it is assumed that it is desired to move the two rods towards each other, this movement takes place against the first spring 3008, corresponding to a first segment having a curve of generally exponential appearance, as in FIG. 29. In contrast, if it is desired to move these two rods apart from each other, this movement takes place against the second spring 4010, thus leading to a second segment of the curve being obtained that is analogous to that of FIG. 31.

In other words, the curve of FIG. 35 does not present a horizontal segment or neutral zone. Thus, starting from the equilibrium position, any mutual movement between the rods takes place against a resistance. This is advantageous since it enables physiological movement to be reproduced faithfully. Eliminating the neutral zone may constitute a therapeutic technique that is most favorable for a patient undergoing arthrodesis.

It should be observed that in the arrangement of FIG. 34, it is possible to adjust various parameters, and thereby impart characteristics of variable resistance. These variable parameters include in particular the distance between the stationary and sliding sleeves, or indeed the stiffness of the springs. Given that the invention lies in the context of arthrodesis, these springs are selected to present high stiffness, so that the two asymptotes of the curve in FIG. 35 are close to the vertical ordinate axis.

Different variants (not shown) of the FIG. 34 arrangement may be provided. Thus, it is possible to eliminate at least one of the two springs, or indeed both of them. Under such circumstances, the sleeve that is no longer associated with a spring is fastened to the link element. Thus, if a single spring is eliminated, then the assembly is rigid in a first direction while it allows damped movement in the opposite direction against the single spring. In contrast, if both springs are eliminated, such that both eyelets are stationary, then the corresponding assembly is hyperstable.

It should be observed that in all embodiments, the springs may be replaced by analogous elements, such as rubber buffers that are capable of sliding on the link element. In addition, when a sleeve is movable relative to a link element, it is advantageous to provide for it to present an axial dimension that is small, like a ring. Under such circumstances, the ring advantageously presents an inside diameter that is greater than the outside diameter of the link member, thus making sliding easier, and that is capable in particular of accommodating bending of the link tube.

FIG. 36 shows an advantageous variant of the invention, based on the arrangement of the preceding figures. However, provision is made to prestress at least one, and in particular both of the springs that oppose movement of the rods. For this purpose, suitable prestress means may be used, such as an assembly constituted by a nut and a lock nut, thereby enabling the prestress to be given a variable value. Thus, the nut and the lock nut may be tightened or loosened along the stroke of the spring.

In other words, at the equilibrium position, corresponding to the origin of the curve, each spring is associated with a respective prestress value. Under such conditions, the curve no longer possesses a horizontal neutral zone as in some of the figures, but rather it possesses a vertical zone referred to as a stable zone ZS. The amplitude of this stable zone corresponds to the magnitude of the force that each rod needs to exert in order to overcome the prestress associated with the respective springs before it is moved.

More precisely, the stable zone ZS comprises two segments ZS₁ and ZS₂, each relating to a respective spring. Given that each spring may be associated with variable prestress, the amplitude of each segment ZS₁ and ZS₂ may thus be adjustable depending on requirements.

In an additional variant of the invention (not shown), the middle zone 1002, interconnecting the two eyelets, may also be of the “external” type. In other words, this zone encompasses or incorporates both eyelets. It is also possible to use a connection of mixed type, i.e. said zone encompasses a single eyelet, on one side only.

FIG. 37 shows an additional variant embodiment of the invention in which the link zone 1102 is made up of a plurality of elements. There is thus a first spring blade 1102₁ referred to as an “inner” blade in that it extends between the adjacent ends of the eyelets 1105. The link zone also includes an outer or peripheral spring blade 1102₂ that extends outside the first spring blade and also both eyelets.

The mechanical link between the two spring blades is provided by any appropriate means, e.g. by a transverse collar 1102₃. Provision may also be made to fill the intervening spaces between the inner spring blade and the outer spring blade, e.g. by means of a damping material. The embodiment of FIG. 37 is advantageous insofar as it enables a single article to be made that acts in two opposite directions, which directions may be prestressed. This embodiment also makes it possible optionally to create a neutral zone, depending on the shape selected for the orifices.

In the preceding examples, the rod forms part of a screw whereas the eyelet forms part of the link element. Nevertheless, it is also possible to devise an inverse configuration, i.e. with the eyelet forming part of the screw while the link element is provided with the rod.

This alternative possibility is shown in FIG. 38 where there can be seen a screw 610 extended by an eyelet 605 defining an orifice 604. A rod 618 extending the link element 602 penetrates in floating manner in this orifice. It should be observed that this rod is bordered by a shoulder 619 of curved profile suitable for co-operating with the walls of the orifice. Under such conditions, when tension is applied, this shoulder comes into contact with the walls of the orifice, allowing mutual hinging between the screw and the link element.

In FIG. 38, the shoulder is placed on the same side of the eyelet as the link element so as to limit any intervertebral approach movement. Nevertheless, as shown in FIG. 39, it is possible to provide for the shoulder to be placed on the opposite side of the eyelet from the link element, thereby serving to limit intervertebral extension movement.

FIG. 40 shows an additional variant embodiment of the invention. There is a rigid eyelet 3005 defining an orifice 3004 together with a rod 3018 penetrating in said orifice. As mentioned above, the eyelet may form part either of the link element or of the vertebral screw, with the rod then belonging respectively to the vertebral screw or to the link element.

The respective dimensions of the orifice and of the rod are such that in the plane of the eyelet there exists a degree of mutual freedom to move in translation in a single direction corresponding to the main direction of the eyelet, which thus forms a slideway. In other words, the rod and the eyelet are constrained to move together in translation in a direction perpendicular to the main direction, i.e. in an up and down direction in FIG. 40. Furthermore, there are only two degrees of freedom in rotation between the rod and the eyelet. Under such conditions, if it is assumed that the rod belongs to a screw that is stationary in the vertebral frame of reference, then any external action such as a movement of the patient causes the rod to move along the slideway until it comes into abutment against the facing wall of the eyelet.

FIG. 41 shows a particular embodiment of the invention, in which the link member is a plate of substantially rectangular shape, given overall reference 2. The length, the width, and the thickness of the plate 2 are given respective references L, [symbol] , and e. In typical manner, L lies in the range 15 mm to 45 mm, [symbol] lies in the range 5 mm to 10 mm, and e lies in the range 1 mm to 8 mm. In addition, the main longitudinal axis of the plate is referenced A.

The plate 2 presents a shape that is inherent thereto, which means that it is capable of maintaining the same shape in the absence of external stresses, in particular under the sole effect of gravity. Furthermore, the shape of this plate does not vary significantly when it is subjected to the stresses that are usual once it has been implanted in a patient.

In this context, the plate may be completely rigid, in which case it is made of metal, for example. Nevertheless, it may present a small amount of flexibility, in a manner analogous to the plate described in U.S. Pat. No. 4,743,260. Under such circumstances, it may be made for example out of a plastics material, a polymer, or indeed a composite material including fibers such as carbon fibers.

The plate 2 is pierced by two oblong orifices 4, each of main axis corresponding with the axis A of the plate. The length and the width of these orifices are written L′ and [symbol] ′, and the distance between the centers of the two orifices 4 is written d. By construction, the length L′ of these orifices is longer than their width [symbol] ′.

The presence of these oblong orifices, and the value of the distance between them, are remarkable characteristics of the plate in accordance with the invention. In contrast, the shape of the plate does not constitute such a characteristic, given that its shape may vary as a function of numerous parameters, in particular of an anatomical type. Thus, by way of example, each plate may present a banana shape, an arcuate shape, or indeed an angled shape. This is shown in FIG. 55 where the plate 2 presents a bend referenced 3.

FIG. 42 shows a pedicular screw 10 for co-operating with the above-described plate 2. In conventional manner, the screw 10 comprises a threaded zone 12 for penetrating into a vertebral body (not shown). Nevertheless, it is possible to make provision to use, not a screw of pedicular type, but rather some other type of vertebral screw.

Thus, the screw may be implanted in the vertebral body, either laterally or anteriorly, in which case it is implanted in the vertebral body by means of a screw thread while leaving a stud projecting outside the vertebra, which stud co-operates with a link element, as described below. The stud may also be supported by a mechanical member other than a screw thread, such as for example a staple or hook placed on the vertebral body and/or the intervertebral bone plates.

The zone 12 is extended by a cylindrical shank 14 that is terminated by a shoulder 16, beyond which there extends a rod 18 of smaller transverse section. The rod 18 is extended by a head 20 that is formed by a portion of a sphere 20₁ truncated by two flats 20₂. The head is also provided in conventional manner with a socket 20₃ for co-operation with a tool (not shown) for the purpose of putting the screw 10 into place in a vertebral body (likewise not shown).

The length of the head is written L″, which length consequently corresponds to the diameter of the spherical portion 20₁. The length L″ is slightly shorter than the length L′ of the orifice 4, while being considerably greater than the width [symbol] ′ of said orifice. In addition, the width [symbol] ″ of the head 20 is slightly less than the width [symbol] ′ of the orifice 4.

FIGS. 43 to 45 show how each screw 10 is put into place relative to the plate 2. The head 20 is initially aligned with the axis of the orifice 4, and then the plate is moved towards it along arrow f₁, so as to cause the head to penetrate through the orifice. This operation is possible given that the length and the width of the head are slightly smaller than those of the orifice 4, as described above.

Thereafter, the screw 10 is caused to turn through about one-fourth of a turn represented by arrow f₂ such that the head 20 then extends transversely across the corresponding oblong orifice 4. Under such conditions, the plate 2 is secured relative to the screw 10 since a first wall (top wall in FIG. 43) of said plate is limited in its movement by the head 20, whereas the opposite wall thereof is limited by the facing vertebral body (not shown).

It should nevertheless be observed that this mutual connection of the plate 2 with the screw 10 is of the “loose” type, i.e. it is accompanied by operating clearance, at least in the absence of external tension acting on the plate and the screw. In other words, the plate 2 is “floatingly” mounted on the screw 10, in the absence of any such tension.

Thus, given that the rod 18 presents dimensions that are smaller than those of the orifice 4, there exists clearance in the two main dimensions of the plate that correspond to its length and its width. In addition, there exists clearance in a third dimension perpendicular to the two above-mentioned main dimensions of the plate. Thus, once implanted, this plate can move a little between the head 20 and the facing vertebral body.

FIGS. 52 to 54 show more precisely the walls of the oblong orifices 4. It can be seen that these walls are rounded, with convex faces facing into the orifices.

In FIG. 52, the walls 4₁ are formed directly in the body of the plate, i.e. there is no additional fitting.

In contrast, in FIGS. 53 and 54, the walls 4₂ and 4₃ are formed by fittings placed in the body of the plate. In this context, these fittings may be made of a material other than the material constituting the remainder of the plate, in particular a metal material. The fitting is then secured to the facing edges of the plate by any suitable means, e.g. by crimping.

In FIGS. 52 and 53, it can be seen that the rounded profiles of the walls are 4₂ are substantially symmetrical about the middle axis of the plate, specifically the horizontal axis. However, in a variant, and as shown in FIG. 14, the walls 4₃ could be asymmetrical relative to such a middle axis.

As can be understood from the description of FIGS. 52 to 54, the respective profiles of the walls of the orifices 2, and of the head 20, are such that they enable the plate to behave like a ball joint relative to the screw when the screw bears against the walls of said orifices. In other words, when the screw has its head 20 bearing against the walls of the orifices 2, there exists at least one, and specifically three degrees of freedom in rotation between the plate and the screw, through an angle range of the order of at least 15 degrees. This imparts a hinged nature to the connection between the plate and the screw, when the plate is put under tension.

FIGS. 46 and 47 show a first variant concerning placing of the plate 2, which plate is to act as a guy. The procedure begins by placing two screws 10 in two adjacent vertebral bodies referenced V₁ and V₂. In this first embodiment, the distance d′ between the free ends of the screws 10 is greater than the distance d between the centers of the orifices 4.

It is then necessary to move these two screws towards each other, e.g. by means of a tool (not shown), so that the above-mentioned distance d′ becomes close to the distance d. Thereafter the plate 2 is moved axially towards both screws so that the heads 20 pass through the orifices 4. The external action exerted by the tool is then released so that the rods 18 press against the furthest-apart walls 4′ of the orifices 4. Finally, each screw 10 is turned through one-fourth of a turn so as to place the heads 20 transversely relative to the orifices 4, as mentioned above with reference to FIGS. 43 to 45.

Once this has been done, the plate 2 exerts forces F₁ and F₂ on the rods 18 of the screws 10. Under such conditions, the plate 2 thus put under tension acts as a guy, opposing the patient being put into kyphosis, i.e. intervertebral flexing. This guy, which engages the articular surfaces one in the other and which puts the anterior portion of the disk and the anterior vertebral ligament under tension, serves to stabilize the intervertebral joint.

FIGS. 48 and 49 show a variant configuration, in which the plate 2 now performs the function of a stay. Unlike the above description, the screws 10 are implanted in the vertebral bodies V₁ and V₂ in such a manner that the distance d″ between them is less than the distance d between the orifices 4. The screws 10 then need to be moved apart from each other, e.g. by means of a tool, so as to increase the value of d″ until it becomes close to d. Thereafter the procedure is as described above, so as to secure the plate 2 to both of the screws 10. After these various steps have been performed, the rods 18 are pressing against the closest-together walls 4″ of the orifices 4. Thus, the plate 2 exerts forces written F′₁ and F′₂ on the pedicular screws 10. Under these conditions, the plate 2 forms a stay, i.e. it opposes the patient being put into lordosis.

Unlike the guy described with reference to FIGS. 46 and 47, the stay as shown in FIGS. 48 and 49 is not stabilizing. It is therefore advantageous for it to be associated with a guy, over the same intervertebral stage, as described in greater detail below.

FIG. 50 shows an additional variant embodiment of the invention, making use of two plates 2′ and 2″ that extend over two vertebral stages. For this purpose, there are initially provided two screws 10′ and 10″ that are analogous to the screws 10 described above, and that are implanted in the furthest-apart vertebrae V₁ and V₃.

Furthermore, a middle screw 10 is implanted in the intermediate vertebral V₂. This screw 110 is provided with two heads 120′ and 120″ placed one behind the other.

For implantation, the first plate 2′ is put into place initially so that the head 20′ of the screw 10′ and the first head 120′ of the middle screw 110 pass through the orifices 4′ formed in the plate. Thereafter, the other plate 2″ is put into place so that the head 20″ of the screw 10″ and the second head 120″ of the intermediate screw 110 pass through the orifices 4″ of this second plate. Finally, the various screws are turned through one-fourth of a turn so as to secure the plates loosely relative to the screws.

The co-operation between the plates 2′ and 2″ and the end screws 10′ and 10″ is analogous to that described above. In addition, in its orifice adjacent to the screw 110, the plate 2′ is interposed between the shank 118 of the first head 120′, and three functional clearances are available, as mentioned above. Finally, the second plate 2″ is interposed between the two heads 120′ and 120″ of the middle screw 110, likewise with functional clearances existing in all three directions of three-dimensional space.

FIG. 57 shows a variant embodiment of the invention in which two plates 2₁ and 2₂ are placed between the same two pedicular screws 110₁ and 110₂, e.g. screws that are analogous to the middle screw 110 described above. For this purpose, each of the screws 110₁ or 110₂ presents two truncated spherical heads, respectively referenced 120₁ and 120′₁, and 120₂ and 120′₂.

The first plate 2₁ is placed in the stay position, as shown in FIG. 49. In contrast, the second plate 2₂ is placed in the guy position, as shown in FIG. 47. These two plates are put into place initially by putting the stay-forming first one of the plates into place as described with reference to FIGS. 48 and 49. Then the free ends of the two screws are moved towards each other so as to enable the guy-forming, second plate to be forced into place.

The embodiment of FIG. 57 is advantageous since it provides an assembly with hyperstability, but nevertheless without inducing high levels of stress on the pedicular screws. In addition, the effectiveness of the guy is doubled by a lever effect, insofar as the stay that is initially put into place provides a thrust point and enables the guy to combine its effect with a lever arm that acts on the bony portion of the implant. This improves imparting a vertebral hollow-back shape, and also improves the stability of the intervertebral joint.

In FIG. 57, each of the two screws 110₁ and 110₂ possesses only two truncated spherical heads. Nevertheless, it is possible for one and/or the other of these screws to be provided with three such heads. This makes it possible firstly to provide a combined guy and stay assembly as shown in FIG. 57. This also makes it possible to connect one and/or the other of these screws to an additional pedicular screw (not shown) by means of an additional plate extending towards another intervertebral stage.

FIG. 50 shows the connection between three adjacent vertebrae. It is naturally possible to connect together some larger number of vertebrae, by using various plates in accordance with the invention. Furthermore, provision can be made to use two plates located on the left and the right when making the connection between two adjacent vertebrae, i.e. one plate on either side of a middle axis that is vertical, assuming the patient is in a standing position.

It should be observed that the invention relates not only to a plate 2 as such, i.e. an extra-discal element for providing intervertebral stabilization, but also to a set of such plates. During the operation, the surgeon has a plurality of plates available that are of different lengths, with the distances between their orifices 4 also being different, in proportional manner. Thus, depending on which vertebral stage is involved, and also depending on the pathology being treated, the surgeon is in a position to select an appropriate inter-orifice spacing. This spacing corresponds to the distance d referenced in particular in FIGS. 41, 46, and 48.

The distance between the orifices and the plates may also be selected in situ, by using mechanical means for enabling said distance to be adjusted. Thus, in FIG. 56, the plate 2″' includes a turn-buckle 5 that co-operates with two rods 7₁ and 7₂, themselves terminated by extensions 9₁ and 9₂, each having a corresponding oblong orifice 4₁₀ or 4₂₀ formed therein.

This embodiment is advantageous insofar as a single article can then present a shape that is variable. This also makes it possible to vary the distance between the orifices in situ in an anesthetized patient, e.g. while under radiographic observation. In a variant, other mechanical means analogous to the turn-buckle shown may be provided, such as a slide bar.

Furthermore, the invention relates to an intervertebral stabilization assembly that comprises at least one plate 2, 2′, and/or 2″, together with at least two screws suitable for co-operating with the or each plate. Generalizing on the basis of the above examples, when n vertebrae are connected to one another, use is made of n pedicular screws, together with (n-1) plates. In this context, the intermediate pedicular screws, i.e. the screws that are not located at the ends, may for example be provided with two swellings like the screw 106. In addition, it is possible to provide this stabilization assembly on one side only of the vertebral column, and it is also possible to provide two such assemblies one on either side of the middle vertical axis of said column.

FIG. 51 shows an additional variant embodiment that is particularly suitable for use with instability due to the absence of an articular surface. There can thus be seen a plate 52 extending in crossed manner between a first pedicle P₁, specifically the left pedicle, of a first vertebra V₁, specifically an upper vertebra, and the other pedicle P₂, specifically the right pedicle, of an adjacent vertebra V₂, specifically the immediately lower vertebra. The plate possesses two end orifices 54 enabling it to co-operate with pedicular screws 60₁ and 60₂ as described above with reference to the first embodiment. In addition, there are two additional plates 61 and 61′ each extending on a respective side of the column. The plate 61 connects together the screws 60₁ and 60′₂, while the plate 61′ connects together the screws 60′₁ and 60₂, as described above with reference to the first embodiments.

The plate 52 advantageously includes a central hinge 53, implemented in any suitable way, giving the plate an angled shape, specifically projecting rearwards. This hinge also enables the distance between its orifices to be defined, and it is capable of being locked rigidly once the appropriate shape and position have been obtained. Such a crossed plate may be put into place so as to act as a guy. In a variant, it is possible to use two plates that are hinged in this way, one acting as a guy and the other as a stay, for use in treating complex deformations, such as rotatory intervertebral dislocations.

Advantageously, the various plates may be put under tension in the guy direction, as shown in FIG. 47. Under such conditions, the assembly of these plates tends to oppose intervertebral flexing of the patient.

Nevertheless, provision may advantageously be made to put some of the plates under tension so that they act as stays, in particular in association with scoliosis. Nevertheless, it should be observed that for a given intervertebral stay, if one of the plates forms a stay on one side, the facing plate will be in a guy position whether on the same side and/or on the other side, in order to provide stability.

FIG. 58 shows a variant embodiment of the invention relating to the structure of the pedicular screw. This screw is given reference 210 and it differs from the screw 10 in that it does not have a head 20. Thus, it possesses a rod 218 suitable for penetrating in an orifice 4 of the plate 2, which rod is pierced by a through opening 219 suitable for receiving a cotter pin 220.

An additional embodiment variant of this pedicular screw is shown in FIG. 59 where the rod 318 of the screw 310 forms a bolt 319 suitable for co-operating with a nut 320. In these alternative embodiments of FIGS. 58 and 59, functional clearances continue to be present in all three directions of three-dimensional space between the screw 210 or 310 and the facing walls of the plates 2.

FIG. 60 shows an additional variant embodiment of the stabilizer element in accordance with the invention, given overall reference 102. This element comprises a rigid body formed by two plate segments 102₁ and 102₂, each made for example out of the same material as that constituting the plate 2 of the first embodiment. In addition, the two segments are pierced by two oblong orifices 104₁ and 104₂ that are analogous to the orifices 4 of the first embodiment.

These two segments 102₁ and 102₂ are separated by a damper buffer 103, e.g. made of elastomer or any other equivalent material. Such a buffer may be replaced by an equivalent damper member, such as a spring.

The connection between the middle buffer 103 and the two segments 102₁ and 102₂ is provided by any appropriate method. Co-operation between the orifices 104₁ and 104₂ and the pedicular screws (not shown in FIG. 60) is performed in a manner analogous to that described with reference to the preceding figures.

FIG. 61 shows an advantageous variant of the invention making use of two plates 102′ and 102″ that are analogous in structure to the plates 102 of FIG. 60. These two plates extend between the same two pedicular screws, and they act respectively as a stay and as a guy, as explained with reference to FIG. 57.

In this context, provision may be made to confer various types of property to the resilient buffers 103′ and 103″. Thus, these two buffers may be designed to be suitable for working both in compression and in traction.

As an alternative, the buffer 103′ of the plate 102′ that acts as a stay may be made so as to be suitable for working in compression only, while the other buffer 103″ forming part of the plate 102″ acting as a guy is then suitable for working only in extension. As a result, use is made of two different dampers, each suitable for working in one direction only. This makes it possible to provide dampers that are very simple, and consequently that are economically advantageous.

As an additional variant, one only of the buffers need be double-acting, while the other one is single-acting. Provision may also be made to use only one plate with a damper, the other plate being rigid.

FIG. 62 shows an additional variant embodiment of the stabilizer element in accordance with the invention, given overall reference 202. This element comprises firstly a rigid body 202₁ in the form of a plate analogous to the plate 2 of FIG. 41. This plate 202₁ is extended by at least one, and specifically by two, end rims 202₂ that extend substantially perpendicularly to the plane of the plate 202₁.

There can also be seen two pedicular screws 410, which extend through orifices 204 that are formed in the plate 202₁. Each screw 410 has a rod 418 with its free end being threaded so as to co-operate with a ball 420 that is pierced by a tapped bore suitable for co-operating with the thread.

In order to secure the stabilizer element 202 loosely relative to each of the screws 410, each rod 418 is initially inserted through the corresponding orifice 204. Thereafter, each elastomer ball 420 is screwed on, thereby serving to retain the element 202 since each ball presents a diameter that is greater than the dimensions of the orifice 204, at least in terms of its width.

Each ball 420, made of a damping material, such as an elastomer, is suitable for coming into abutment against the facing walls of the rims 202₂. This embodiment is advantageous insofar as the presence of the balls 420 serves to damp the various movements to which the connection assembly in accordance with the invention is subjected. The purpose of this plate is to limit movement to a selected sector of rotation, while ensuring that the end of movement is damped.

In the embodiment described, there are two damper balls 420. Nevertheless, in a variant, it is possible to make use of only one such ball 420. Under such circumstances, the other rod 418 co-operates with the facing walls of the plate 202₁ in the same manner as one or other of the above-described embodiments.

FIG. 63 shows an additional variant embodiment of the invention. In FIG. 63, mechanical elements that are analogous to those of FIG. 62 are given the same reference numerals, plus 100.

Firstly there is a stabilizer element comprising a plate 302₁ having end rims 302₂ projecting therefrom. A central block 302₃ is also provided that defines abutment walls 302₄ that face the rims 302₂. In addition, the pedicular screws 510 are analogous to the screws 410 of FIG. 62.

In service, the damper balls 520 are thus likely to come into abutment either against the rims 302₂ as in the example of FIG. 62, or else against the walls 302₄ of the intermediate block. The damping provided in this way thus acts in two opposite directions. This embodiment makes it possible to allow for free movement over an advantageous sector of rotation, while terminating said movement by damping at each end.

In a variant, as in FIG. 62, provision may be made for the block 302₃ to extend axially over a shorter distance, so that it constitutes only one abutment wall for only one damper ball 520. Under such circumstances, the other pedicular screw may be analogous to the screw 10, 210, or 310, of preceding figures.

In FIGS. 60 to 63, there can be seen various damper elements. As mentioned above, these elements are nevertheless relatively stiff, so as to damp only micromovements and to absorb vibration.

FIGS. 64 to 66 show an additional variant of the invention.

In this embodiment, the pedicular screws 610 are analogous to the screw 10, except concerning the shape of their heads 620. The heads are spherical in shape without being truncated by flats, as shown in FIG. 42 in particular. In addition, each oblong orifice 404 of the plate 402 has a main portion 404₁ that substantially defines a circle of diameter that is slightly greater than the diameter of the head 620. This middle portion is extended by two axial notches or ears 404₂ that extend away from opposite sides of the middle portion 404₁ along the main axis of the plate.

For assembly purposes, and as shown in FIG. 64, the plate is initially brought up to the screw so that the spherical head can pass through the circular main portion. Thereafter, as shown in FIGS. 65 and 66, each screw is moved laterally so as to become received in a corresponding notch. It should be observed that depending on whether the plate acts as a guy or as a stay, the screw is received either in the notch going towards the free edge of the plate, or else in the notch going towards the middle of the plate.

Once assembly has been performed, the walls of one or other of the notches co-operate with the facing walls of the screw, i.e. the walls of the head 620 and of the rod 618 forming part of the screw. As in the above-described embodiments, these various walls are suitable for allowing at least one, and preferably three, degrees of freedom to exist in rotation between the plate and the rod. Consequently, co-operation between these two elements takes place in hinged manner.

This hinging possibility is shown in FIG. 67, which shows one possible configuration, making use of a screw 710 analogous to the screw 610 of FIGS. 64 to 66 and presenting a plurality of spherical heads 720₁, 720₂, and 720₃. This screw is also provided with a first rod 718₁, and with two intermediate rods 718₂ and 718₃.

In the example of FIG. 67, there can be seen a first plate 402₁ in the vicinity of the vertebral bodies (not shown), which plate is mounted to act as a stay. Thereafter, there is a second plate 402₂ connecting together the same pedicular screws as the first plate, but mounted to act as a guy. Finally, a third plate 402₃ extends from the pedicular screw 710 towards another screw (not shown) belonging to another intervertebral stage. This configuration ensures that hinging is possible in all three dimensions between each plate and the screw.

The invention is not limited to the embodiments described and shown.

Thus, the various figures show pedicular screws, i.e. screws that are associated with posterior link elements. Nevertheless, it is also possible to use screws of some other type, which screws are implanted from the front of the vertebral column. Under such conditions, the link element that connects them together is of the anterior type, which is particularly advantageous for the cervical column.

In addition, provision can be made for a given vertebral stage to be connected together both by an anterior link element and by a posterior link element. Under such conditions, one of these elements acts as a stay and the other as a guy during a first type of patient movement, whereas when the patient performs a movement of the opposite type, the said one acts as a guy and the other as a stay.

In the embodiment of FIGS. 24, 25, et seq., the rigid tube 1002 connects together the two link sleeves 1006 on the same side of the eyelets 1005, specifically on the left of the eyelets in the figures. Nevertheless, provision can be made for the tube to extend obliquely, i.e. from one side of a first eyelet to the opposite side of the second eyelet. This is shown in FIG. 68, where the rigid tube connects the left side of the upper eyelet to the right side of the lower eyelet. This is advantageous in terms of force distribution, in that it avoids subjecting the link element to bending stress. This thus makes it possible to reduce unwanted deformation, and to limit weakening by shear.

The invention makes it possible to achieve the above-specified objects.

In this respect, it should initially be emphasized that it is to the merit of the Applicant to have identified the causes of the drawbacks in the prior art.

The Applicant has observed that prior art systems can be improved insofar as they are of the one-piece type and they extend regionally over the entire vertebral column.

Thus, the state of the art makes use firstly of one-piece type fastening between the pedicular screws and the plate that connects them together. Under such conditions, the bending and tearing-out forces that act on the plate, after it has been implanted, are transmitted directly to the screws, which therefore tend to move relative to the vertebral body. These high stresses are exerted most particularly on the pedicular screws that are to be found that the ends of the assembly.

The above-described defects are even more severe when the quality of the bone is poor, as with osteoporosis, or when the bone is naturally weaker, as in the vicinity of the sacrum. This explains in particular why these phenomena of screws being torn out or indeed of screws moving in the bone, are more troublesome with any regional and one-piece assembly that goes down as far as the sacrum.

Furthermore, prior art plates are regional, i.e. a single plate connects together more than two vertebral stages. The Applicant has observed that such a configuration does not provide a satisfactory solution in terms of positioning the vertebrae relative to one another.

By their very nature, such surgical assemblies are intended to stabilize the vertebral column in a good position. Such stabilization then allows appropriate bone fusion to be obtained, known as arthrodesis. A favorable position, in particular a hollow-back curve, gives rise to satisfactory economy in the upright human position, giving rise to physiological muscle work without contracture type dysfunctioning.

However, with a regional assembly as provided in the prior art, it is necessary to curve the rod or the plate used in order for it to be rounded and to impart the desired hollow-backed shape to the vertebral column. In this respect, it should be observed that this shape is initially diminished because of the pathology. Thus, the vertebral column adapts poorly to the curved plate or rod because of the posterior positions of the centers of rotation between the adjacent vertebrae.

In contrast, the present invention makes use of functional clearances between the screws and the link element. This makes it possible to obtain hinges between the screws and said element, thereby avoiding mechanical stresses at the screw/bone interface.

Also known in the prior art is hinging by equatorial capture between each screw and the link element. However, unlike the subject matter of the present invention, such hinging is associated with a single center of rotation, and is thus a kind of ball-and-socket connection. In contrast, the present invention makes much more complex hinging possible, better approximating natural physiological movement.

Thus, it is known that the intervertebral joint does not possess a single center of rotation, but rather a cloud of centers of rotation. In other words, reproducing the physiological joint needs a plurality of instantaneous centers of rotation to be taken into consideration, rather than a single permanent center of rotation. Under such conditions, the joint created by the invention presents better mechanical aptitude for accompanying vertebral movement than does a hinge that possesses only a single fixed center of rotation, as in the above-described prior art.

In addition, the vertebral column has a plastic quality continuously seeking equilibrium, such that the centers of rotation are likely to vary over the lifetime of the patient as a result of the component elements of the column becoming deformed. This makes it necessary to avoid imposing an a priori center of rotation. Were that to be done, a conflict could arise that would be the source of iatrogenic pathology.

It should be observed that the prior art offers the possibility of varying the positioning between the pedicular rods. Nevertheless, this position is adjusted a priori in such a manner that after adjustment the hinging between the two screws and the link element is of the one-piece type. In operation, relative movement, if any, is made possible solely by the structure of the link element, e.g. because it incorporates a damper. This should be compared with the arrangement of the invention that, as described above, continuously offers the possibility of relative movement associated with a cloud of centers of rotation.

Furthermore, the present invention relies advantageously on the notion of segmentation. In other words, a given plate connects together only two adjacent vertebrae, thereby enabling various pathologies to be treated more effectively. In particular, it is emphasized that the use of a plurality of successive plates makes it possible to impart a satisfactory degree of hollow-back curvature, that is not to be found in the prior art using a single plate.

Thus, the advantageously segmented assembly of the invention, as compared with regional solutions of the prior art, makes it possible in particular to obtain the exact looked-for degree of hollow-back curvature by appropriately selecting inter-pedicular distances. As a result, by virtue of the invention, each intervertebral joint is considered individually and is treated as a function of its own characteristics, with this taking place all along the vertebral column.

As set out in the introduction to the present application, the arthrodesis stabilizer assembly in accordance with the invention permits relative movement in rotation between two vertebrae that is less than or equal to about 10% of the natural physiological movement. Nevertheless, the various embodiments described above can be subdivided into two categories.

Thus, there are firstly assemblies that allow no movement in rotation between the two vertebrae they connect together. This applies in particular to the arrangement of FIG. 34 when there is no spring and all four sleeves are stationary, to the arrangement of FIG. 36 when the prestress of the springs is not exceeded, or indeed the arrangement of FIG. 57. In contrast, in certain other embodiments, angular movement between the vertebrae is permitted. This applies in particular to the assembly of FIG. 34 with the spring, or indeed to the assemblies of FIGS. 60 and 61.

In accordance with the invention, provision may be made to fit certain vertebral stages with assemblies that are hyperstable, thus not allowing any movement, and to fit other vertebral stages with assemblies that allow a small amount of movement. It is also possible to fit still other vertebral stages with prosthesis type arrangements, that permit movements that are greater than or equal to 50% of the natural physiological movement. These assemblies, which are not in accordance with the invention, may for example be of the type described and claimed in the French patent application filed on the same day as the present patent application by the same Applicant and entitled “Ensemble extradiscal de stabilisation prothétique intervertébrale” [An extra-discal assembly for vertebral prosthetic stabilization].

By way of example, it is possible to fit the L₅-S₁ stage with a prosthesis, the L₄-L₅ stage with an arthrodesis assembly that allows a certain amount of angular movement, and the L₃-L₄ stage with a prosthesis. In certain other cases, it is possible to fit the L₅-S₁ stage with an arthrodesis assembly that allows a small amount of movement, the L₄-L₅ stage with a hyperstable arthrodesis assembly, and the L₃-L₄ stage with a prosthesis. When there is scoliosis, it is possible to fit the stages D₁₂ to L₄ with a hyperstable arthrodesis assembly, the stage L₄-L₅ with an arthrodesis assembly permitting a small amount of angular movement, and finally the L₅-S₁ stage either with a prosthesis assembly, or with an arthrodesis assembly that allows a small amount of movement.

Finally, provision can be made to fit a single vertebral stage with two different assemblies. By way of example, these two different assemblies may be two arthrodesis assemblies in accordance with the invention, one of which is hyperstable and the other of which allows a small amount of angular movement. In addition, provision can be made for a first assembly to correspond to a prosthesis, while the other assembly is of the arthrodesis type, with or without angular movement being possible.

1-17. (canceled)

18. An extra-discal intervertebral stabilization assembly for arthrodesis, the assembly comprising:
at least two vertebral screws suitable for penetrating into two different vertebrae; and
a link member suitable for connecting the two screws together;
one of the link member or each screw possessing a rod, while the other of the link member or each screw is provided with at least one eyelet having walls that present a shape that is inherent thereto, said walls defining an orifice, the or each rod being suitable for penetrating in the or each orifice, with freedom to move in at least one direction of the plane of said orifice.

19. An assembly according to claim 18, wherein movement is possible in two mutually perpendicular directions in the plane of the orifice, the rod and the walls of the eyelet forming a hinge only when that one of the rod and the eyelet that is carried by the link member puts under tension that one of the eyelet or the rod that is carried by the screw.

20. An assembly according to claim 19, wherein said hinge acts via a single point of contact between the rod and the walls of the eyelet.

21. An assembly according to claim 19, wherein said hinge acts via a contact of the flat-on-flat type, so as to make sub-luxation possible.

22. An assembly according to claim 18, wherein the rod is free to move relative to the walls of the eyelet in a single direction in the plane of the eyelet, so as to form a slideway connection between the rod and the eyelet.

23. An assembly according to claim 18, wherein at least one eyelet presents rigid walls.

24. An assembly according to claim 18, wherein at least one eyelet presents a wall that is deformable, at least in places, under the effect of a stress that is considerably greater than that due to gravity.

25. An assembly according to claim 18, wherein at least one eyelet is provided with means for resisting movement of the rod.

26. An assembly according to claim 25, wherein the means for resisting movement comprise a deformable narrowed wall of the eyelet.

27. An assembly according to claim 25, wherein the means for resisting movement comprise a partial filling of the orifice defined by the eyelet by means of an elastomer material.

28. An assembly according to claim 25, wherein the link member comprises an elongate body and two sleeves suitable for being fitted on said body, each sleeve being provided with a corresponding eyelet.

29. An assembly according to claim 28, wherein a first sleeve is stationary relative to the body, while a second sleeve is movable relative to the body.

30. An assembly according to claim 29, wherein at least one spring is provided interposed between the movable sleeve and the stationary sleeve, and/or between the movable sleeve and an end abutment of the elongate body.

31. An assembly according to claim 25, wherein the link member is formed by a single link element presenting two eyelets, each eyelet being suitable for receiving a rod carried by a vertebral screw.

32. An assembly according to claim 25, wherein the link member is formed by two distinct link elements, each link element being provided with two eyelets, each rod of a vertebral screw being suitable for penetrating in two successive eyelets, carried respectively by the two distinct link elements.

33. An assembly according to claim 32, wherein a first spring is interposed between the movable sleeve and the stationary sleeve of a first link element, while a second spring is interposed between the movable sleeve and the end abutment of the second link element.

34. An assembly according to claim 25, wherein when the rod bears against an axial end of the eyelet, the main axis of the rod being perpendicular to the plane of the eyelet, the screw and the eyelet define a free zone that is not occupied by the rod, said free zone presenting a dimension along the main axis of the eyelet that is greater than or equal to 50%, in particular greater than or equal to 100%, of the dimension of the rod measured along the same main axis.

35. An assembly according to claim 30 wherein a first spring is interposed between the movable sleeve and the stationary sleeve of a first link element, while a second spring is interposed between the movable sleeve and the end abutment of the second link element.
